(19) United States
(12) Patent Application Publication
(10) Pub. No.: US 2024/0050420 A1
(43) Pub. Date: Feb. 15, 2024

(54) NANOMEDICINES FOR TREATMENT OF DISEASE

(71) Applicant: Chapman University, Orange, CA (US)

(72) Inventors: Surya Nauli, Orange, CA (US); Rajasekharreddy Pala, Orange, CA (US)

(21) Appl. No.: 18/222,775

(22) Filed: Jul. 17, 2023

Related U.S. Application Data
(60) Provisional application No. 63/390,131, filed on Jul. 18, 2022.

(51) Int. Cl. A61K 31/445; A61K 9/51; A61K 31/436; A61K 47/55; A61P 13/12

(52) U.S. Cl. CPC A61K 31/445; A61K 9/5146; A61K 31/436; A61K 47/551; A61P 13/12

(57) ABSTRACT

Disclosed herein are nanoparticulate formulations of pharmaceutical agents and methods of treating kidney diseases with the formulation.

### CROSS-REFERENCE TO RELATED APPLICATIONS

The present application claims the benefit of U.S. Provisional patent application 63/390,131 filed Jul. 18, 2022, the entire contents of which are incorporated by reference herein.

### STATEMENT REGARDING FEDERALLY SPONSORED RESEARCH

This invention was made in part with government support under Grant/Contract Number HL147311 awarded by the National Institutes of Health. The government has certain rights in the invention.

### BACKGROUND

Polycystic kidney disease (PKD) is characterized by abnormal cellular proliferation in renal epithelial cells resulting in cyst growth. The mammalian target of rapamycin (mTOR) signaling pathway is involved in proliferation, and mTOR and its downstream signaling molecule S6K are highly expressed in human PKD kidneys. The use of rapamycin to inhibit mTOR signaling has subsequently shown to be promising in many animal studies. Daily intraperitoneal injection and oral dosages of rapamycin have been used with a range of 0.2-5 mg/kg and 2-100 mg/kg, respectively. The overall consensus from the animal studies is that mTOR inhibition reduces kidney weight, inhibits renal cyst growth and/or preserves renal function in many different animal models for PKD including in Pkd1 and Pkd2 knockout mice. Unfortunately, large randomized clinical trials demonstrated unimpressive results with no improvement in total kidney volume and/or kidney function. Aside from the side effects, it is thought that the dosing is suboptimal in these human trials.

Suboptimal dosage with already high side effects could ideally be managed with an advanced kidney-targeted drug delivery technology to reduce side effects while increasing drug efficacy. Nanotechnology has been used for kidney delivery systems. In fact, nanoparticles (NPs) have been proposed to be the future therapy of PKD. These NPs were loaded with either microRNA or metformin to lower cyst burden (Wang et al. J Control Release 329:1198-1209, 2021; Tsai et al. Adv Healthc Mater 8:e1801358, 2019). While these NPs have been used to deliver cargos to the kidney, a more specific kidney-targeting NPs has yet to be generated. This kidney-specific target can ideally be achieved through folate conjugation.

Drug-folate conjugation has been proposed, and folate conjugation results in polycystic kidney-specific targeting (Shi et al. J Control Release 293:113-125, 2019). The concept of folate conjugation is based on the discovery that the cells lining renal cysts express the folate receptor at significantly higher levels in PKD mouse models compared to kidneys from wild-type mice (Shillingford et al. J Am Soc Nephrol 23:1674-1681, 2012). When folate is conjugated with rapamycin, for example, folate-rapamycin interacts with folate receptors resulting in intracellular uptake via folate-receptor-mediated endocytosis. This concept should promote a high selectivity for polycystic kidneys, although folate-rapamycin and rapamycin alone show similar effectiveness to treat polycystic kidneys (Kipp et al. Am J Physiol Renal Physiol 315:F395-F405, 2018). The use of folate-conjugated antioxidant therapy has also been demonstrated in kidneys (Knight et al. J Am Soc Nephrol 23:793-800, 2012). 4-hydroxy-[2,2,6,6-tetramethylpiperidin-1-yl]oxidanyl (4-hydroxy-TEMPO or TEMPO) is an antioxidant that scavenges free radicals, and it has been shown to alleviate kidney injury and fibrosis by reducing renal superoxide.

Thus, disclosed herein are folate-conjugated NPs loaded with rapamycin and/or TEMPO to treat polycystic kidneys.

### SUMMARY

Disclosed herein are methods of targeting one or more pharmaceutical agents to an organ of a subject in need thereof, comprising administering to a subject in need thereof a nanoparticulate formulation, the nanoparticles comprising one or more pharmaceutical agents encapsulated in a polymer. In some embodiments, the pharmaceutical agent is selected from the group consisting of an mTOR inhibitor, an antioxidant, a phosphatidylinositol-3 kinase-related kinases (PIKKs) inhibitor, an ATP-competitive mTOR kinase inhibitor, an mTOR/P13K dual inhibitor, and an mTORC1/mTORC2 dual inhibitor. In some embodiments, the pharmaceutical agent is selected from the group consisting of rapamycin, a rapalog, temsirolimus, everolimus, ridaforolimus, umirolimus, zotarolimus, torin-1, torin-2, vistusertib, dactolisib, voxtalisib, BGT226, SF1126, AZD8055, AZD2014, OSI-027, INK-128, MLN0128, VX970, NVP-BEZ235, AZ20, AZ31, PKI-587, 4-hydroxy-[2,2,6,6-tetramethylpiperidin-1-yl]oxidanyl (4-hydroxy-TEMPO or TEMPO), metformin, salsalate, oxypurinol, cincalcet, GLPG2737, benzbromarone, niclosamide, senicapoc, pioglitazone, 2-deoxy-D-glucose, a SGLT2 inhibitor, pemafibrate, bardoxolone, sulforaphane, probucol, emalipretide, valproic acid, nicotinamide, and tolvaptan. In some embodiments, the organ is selected from the group consisting of kidney, lung, ovary, pancreas, and vascular system.

Also disclosed herein are nanoparticulate formulations of pharmaceutical agents for the treatment of chronic kidney diseases, the nanoparticles comprising one or more pharmaceutical agents encapsulated in a polymer.

In some embodiments, the pharmaceutical agent is an mTOR inhibitor. In some embodiments, the pharmaceutical agent is rapamycin. In some embodiments, the nanoparticles further comprise an antioxidant pharmaceutical agent. In some embodiments, the nanoparticles further comprise the pharmaceutical agent 4-hydroxy-[2,2,6,6-tetramethylpiperidin-1-yl]oxidanyl. In some embodiments, the nanoparticles comprise rapamycin and 4-hydroxy-[2,2,6,6-tetramethylpiperidin-1-yl]oxidanyl encapsulated in a polymer.

In some embodiments, the polymer is a triblock copolymer. In some embodiments, the triblock copolymer is a poloxamer. In some embodiments, the poloxamer is poloxamer 188.

In some embodiments, the one or more pharmaceutical agents are conjugated to folate. In some embodiments, one of the one or more pharmaceutical agents are conjugated to folate. In some embodiments, more than one of the one or more pharmaceutical agents are conjugated to folate.

Also disclosed herein are methods for treating a chronic kidney disease comprising administering to a subject in need thereof a nanoparticulate formulation disclosed herein. In some embodiments, the chronic kidney disease is polycystic kidney disease. In some embodiments, the nanoparticulate formulation is administered less frequently than daily. In some embodiments, the nanoparticulate formulation is administered weekly.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIGS. 1A-I depict synthesis and characterization of polycystic kidney targeted nanoparticles. FIG. 1A: Schematic representation of synthesis, drug loading and surface functionalization of PLGA nanoparticles. FIG. 1B: The morphological features of the plain (unloaded) and drugs-loaded PLGA nanoparticles were imaged with scanning electron microscopy. Scale bars, 200 nm. FIG. 1C: Hydrodynamic size distribution of unloaded and drugs-loaded nanoparticles. FIG. 1D: Average zeta-potential measurements of unloaded and drugs-loaded nanoparticles. FIG. 1E: Average hydrodynamic size stability measurements of nanoparticles in saline (PBS), mouse plasma and growth media (DMEM) containing fetal bovine serum. FIG. 1F: Measurement of average amount of rapamycin and TEMPO loading into PLGA nanoparticles expressed in % weight ratio. FIG. 1G: Drug release kinetics from the PLGA nanoparticles in a growth media with serum for 7 days. FIG. 1H: Folate receptor expressions by immunohistochemistry on kidney sections from wild-type vs Pkd2 mice. FIG. 1I: Average folate receptor (FR) expression in the kidney tissues by western blot analysis. N=3 for all experiments. ***, P<0.001. Statistical analysis was performed using ANOVA followed by Tukey's multiple comparison test.

FIGS. 2A-C depict functional nanoparticle-targeting in 3-dimensional culture. FIG. 2A: Human renal epithelia from normal kidney (NK) cells and autosomal dominant polycystic kidney disease (ADPKD) were grown to form cysts in a 3D Matrigel system with collagen in the presence of forskolin and EGF for 1 day followed by different treatments for 5 additional days. All representative live DIC and fluorescence images were taken at 6 days. Control treatment was denoted as vehicle (saline-only). 3D non-cyst/cyst cultures were imaged for nanoparticles uptake, cyst formation and cysts size measurements. The inset images at the right showing the enlarged cystic area taken from the white square boxes selected from the images at the left. Scale bar, 200 μm. FIG. 2B: Percent number of cysts were measured and plotted in bar graphs; each point represents an averaged of replicate. FIG. 2C: Cyst diameters were measured in each well. ****P<0.0001 compared to wild-type vehicle. #P<0.05, ###P<0.001, ####P<0.0001 compared to Pkd2 vehicle. Statistical analysis was performed using ANOVA followed by Tukey's multiple comparison test.

FIGS. 3A-D depict in vivo fluorescence imaging of nanoparticles. FIG. 3A: Dorsal in vivo fluorescence imaging of wild-type (WT) and Pkd2 mice injected intravenously with either unconjugated (non-folate) or folate-conjugated (folate) labeled nanoparticles (NPs) for seven days. Fluorescence images of dissected organs. FIG. 3B: Quantitative biodistribution of NPs by total fluorescence measurements of the whole body. FIG. 3C: Fluorescence intensity quantitative analysis in kidneys among different groups. FIG. 3D: Comparative quantitation's by fluorescence intensity among different organs. N=5 for all experiments. *P<0.05, **P<0.01, ***P<0.001, ****P<0.0001 compared between WT (non-folate NPs) and WT (folate NPs). #P<0.05, ##P<0.01, ###P<0.001, ####P<0.0001 compared between Pkd2 (non-folate NPs) and Pkd2 (folate NPs). Statistical analysis was performed using ANOVA followed by Tukey's multiple comparison test.

FIGS. 4A-D depict bioavailability and functionality of nanoparticles. FIG. 4A: The mean concentration-time profile of rapamycin and TEMPO from lysates of different organs. Drug-NPs were administered intravenously once, while drug-only was intravenously injected daily. N=5. FIG. 4B: Immunoblotting study shows the effects of different drugs, and drug loaded NPs on mTOR signaling pathway in kidney lysates using the surrogate downstream markers phosphorylated-S6K (p-S6K) and phosphorylated-S6 (p-S6). Total-S6K (t-S6K), total-S6 (t-S6) and equal loading control (GAPDH). N=3. FIG. 4C: Immunoblot data for p-S6K in bar graphs. FIG. 4D: Immunoblot data for p-S6 in bar graphs. ****P<0.0001 compared with wild-type (WT) mice. #41p<0.01 and ####P<0.0001 compared with Pkd2. Statistical analysis was performed using ANOVA followed by Tukey's multiple comparison test.

FIGS. 5A-G depict kidney-targeted nanoparticles to inhibit renal cyst, improve body weight and survival. FIG. 5A: Kidney comparison of wild-type (WT) and Pkd2 kidneys after 9-weeks of treatment. Color, brightfield, fluorescent images were captured consecutively. The hematoxylin and eosin (H&E) sections were obtained from the same corresponding kidneys.

FIG. 5B: Fluorescence intensity measurements from isolated kidneys were normalized to total kidney volume (TKV). FIG. 5C: Body weight of 9-weeks-old mice was reduced by daily rapamycin use. FIG. 5D: Total kidney weight measurement. FIG. 5D. Percent weight ratio of 2 kidneys to body weight (2 KW/BW). FIG. 5F: Total cyst number calculated using H&E staining. FIG. 5G: Kaplan-Meier survival curves analyzed with Log-rank test. N=5 for all experiments. *P<0.05, **P<0.01, ***P<0.001, ****P<0.0001 compared to wild-type vehicle. #P<0.05, ##P<0.01, #44P<0.001, #44t #P<0.0001 compared to Pkd2 vehicle. Statistical analysis was performed using ANOVA followed by Tukey's multiple comparison test.

FIGS. 6A-G depict drug-loaded nanoparticles improving renal structure and function. FIG. 6A: Hematoxylin and eosin (H&E) staining of different groups of kidneys. FIG. 6B: Percentage cyst index and average cyst sizes after 9 weeks of indicated treatments. N=5 to 13.

FIG. 6C: Masson trichrome staining of different groups of kidneys showing normal kidneys (dark-red) or cystic kidneys with fibrosis or collagen deposition (purple-blue). Scale bar, 200 μm.

FIG. 6D: The percent fibrosis was calculated from the fibrotic area per total cross-sectional area. FIG. 6E. Plasma nitrate measurement. FIG. 6F: Kidney function measurement of blood urea nitrogen (BUN). FIG. 6G: Kidney function measurement of serum creatinine. N=5 for all experiments where not indicated. *P<0.05, **P<0.01, ***P<0.001, ****P<0.0001 compared to wild-type vehicle. #P<0.05, ##P<0.01, ###P<0.001, ####P<0.0001 compared to Pkd2 vehicle. Statistical analysis was performed using ANOVA followed by Tukey's multiple comparison test.

FIGS. 7A-D. FIG. 7A: Zeta-potential measurement (peak at −28.6 mV) for rapamycin-loaded NPs. FIG. 7B: Zeta-potential measurement (peak at −27.8 mV) for TEMPO-loaded NPs.

FIG. 7C: Hydrodynamic size distribution (peak at 209 nm) for rapamycin-loaded NPs. FIG. 7D: Hydrodynamic size distribution (peak at 212 nm) for TEMPO-loaded NPs.

FIGS. 8A-C. FIG. 8A: XPS patterns of PLGA NPs with different surface modifications and drug-loading showing one complete spectra with several more focused survey spectra, including the C 1s, N 1s and O 1s spectra. FIG. 8B: XRD patterns of PLGA NPs with different surface modifications and drug loading. FIG. 8C: FTIR spectra showing the infrared signatures of PLGA NPs with different surface modifications and drug loading.

FIGS. 9A-C. FIG. 9A: Cellular toxicity of nanoparticles was visualized by DIC/fluorescence imaging. Annexin-V (green) and propidium iodide (PI; red) were used as apoptotic and necrotic markers, respectively. FIG. 9B-C: Toxicity was quantified with flow cytometry analysis. Data are tabulated after NP treatment for 48 hours. Negative control (no staining); positive controls (30 min-methanol permeabilization) were stained with PI-only, annexing-V-only or both annexin-V/PI. N=3 for each group.

FIGS. 10A-C depicts renal epithelia treated for 4 hours with either unconjugated NPs (FIG. 10A, non-folate NPs) or folate-conjugated NPs (FIG. 10B, folate NPs). Differential interference contrast (DIC) to indicate the cells; Cy5 fluorescence to show the cellular uptake of NPs. Scale bar, 100 μm. FIG. 10C: The fluorescence intensity (l/μm²) in the time-dependent manner was quantified in cytosol, nucleus and the whole cell (total). N=5 for all experiments. ****P<0.0001 compared between unconjugated and folate-conjugated NPs.

FIG. 11 depicts karyotyping analysis of human epithelial cells from normal kidney (NK) or polycystic kidney (ADPKD). Spectral karyotyping shows somatic chromosomes (1 to 22) with a pair of sex chromosomes (XY). NK cells with normal chromosome numbers and ADPKD cells with polyploidy were shown.

FIG. 12 depicts folate receptor fluorescence and their quantifications were measured in wild-type (WT) and Pkd2 mice using renal-specific FolateRSense 680. The boxes represent the areas for quantitation. N=3. ****P<0.0001 comparing between wild-type and Pkd2 with Student-t test.

FIG. 13 depicts immunohistochemistry images showing the presence of folate receptor in wild-type (WT) and Pkd2 kidney sections and the nanoparticle targeting.

FIG. 14 depicts representative immunofluorescence images of mice kidney sections. Blue (DAPI) stained nucleus, green (DBA) stained proximal tubules or collecting duct, orange showing the selective targeting of NPs to the tubules.

FIGS. 15A-C. FIGS. 15A-B depict the formulation and equilibration of HPLC column and liquid solvent were achieved using known rapamycin and TEMPO concentrations to obtain a standard approach and retention time. An HPLC calibration curve for both TEMPO and rapamycin (reference standards) are also shown. FIG. 15C: HPLC chromatogram of plasma and plasma spiked with an internal standard (Cyclosporin A; IS), TEMPO, and rapamycin.

FIGS. 16A-B. FIG. 16A: Representative images showing the immunofluorescence staining of phospho-ribosomal protein S6 (p-S6) in the mouse kidney sections of different groups. Scale bar, 500 μm. FIG. 16B: A violin plot showing the percentage of cytosolic p-S6 positive cells, normalized to the total DBA-positive nuclei in the renal tubules. N=5 for all experiments. ****P<0.0001 compared to wild-type vehicle. ##4 #P<0.0001 compared to Pkd2 vehicle.

FIG. 17A-B. FIG. 17A: Representative images showing the immunofluorescence staining of cell proliferation marker (Ki-67) in the mouse kidney sections of different groups. Scale bar, 500 μm. FIG. 17B: A violin plot showing the percentage of cytoplasmic Ki-67 positive cells, normalized to the total DBA-positive nuclei in the renal tubules. N=5 for all experiments. *P<0.05, ****P<0.0001 compared to wild-type vehicle. #P<0.05, ####P<0.0001 compared to Pkd2 vehicle.

FIGS. 18A-B. FIG. 18A: Hematology data of different treatments for either 14 or 28 days. FIG. 18B: Wright-Giemsa staining shows the lymphocytes and neutrophils (magnification, ×400) in different treatment groups for either 14 or 28 days. Scale bar, 100 μm. N=5 for all experiments. ****P<0.0001 compared to wild-type vehicle. #P<0.05, ##P<0.01, ##P<0.001, ####P<0.0001 compared to Pkd2 vehicle.

FIG. 19 depicts IL-1β, IL-6 and TNF-α levels from kidney supernatants determined by ELISA and their quantifications were plotted in bar graphs 9 weeks after treatment period. N=5 for all experiments. *P<0.05, **P<0.01, ***P<0.001, ****P<0.0001 compared to wild-type vehicle. #P<0.05, ##P<0.01, ##P<0.001, ####P<0.0001 compared to Pkd2 vehicle.

FIGS. 20A-C. FIGS. 20A-B depict coronal and trans-axial CT imaging of mice at 3, 6 and 9 weeks of treatment time. FIG. 20C: Plots showing the total kidney volume at 3, 6 and 9 weeks of treatment time. N=5 for all experiments. *P<0.05, **P<0.01, ***P<0.001, ****P<0.0001 compared to wild-type vehicle. #P<0.05, ##P<0.01, ###P<0.001, ####P<0.0001 compared to Pkd2 vehicle.

FIG. 21A-B depict systolic blood pressure (FIG. 21A, SBP), diastolic blood pressure (FIG. 21B, DBP), and mean arterial pressure (FIG. 21C, MAP) for during treatment period. N=5 for all experiments. ****P<0.0001 compared to wild-type vehicle. #44/4P<0.0001 compared to Pkd2 vehicle.

FIG. 22 depicts H&E staining of major organs collected from different groups of mice at the end of the 9-week treatment.

### DETAILED DESCRIPTION

We here report a generation of nanoparticles (NP) for to target pharmaceutical agents to specific organs. Specifically disclosed are polymer-encapsulated pharmaceutical agent nanoparticles which improve delivery of the pharmaceutical agents to the kidney and allow less frequent dosing.

Pharmaceutical agents suitable for delivery to an organ according to the disclosures herein include, but are not limited to, mTOR inhibitors, antioxidant, phosphatidylinositol-3 kinase-related kinases (PIKKs) inhibitors, ATP-competitive mTOR kinase inhibitors, dual mTOR/P13K dual inhibitors, mTORC1/mTORC2 dual inhibitors. Non-limiting examples of mTOR inhibitors include rapamycin and rapalogs, temsirolimus, everolimus, ridaforolimus, umirolimus, zotarolimus, torin-1, torin-2, vistusertib, dactolisib, voxtalisib, BGT226, SF1126, AZD8055, AZD2014, OSI-027, INK-128, MLN0128, VX970, NVP-BEZ235, AZ20, AZ31, and PKI-587. Non-limiting examples of PIKK inhibitors include torin-2, AZD8055, AZD2014, OSI-027, INK-128, MLN0128, VX970, NVP-BEZ235, AZ20, and AZ31. Non-limiting examples of antioxidants include 4-hydroxy-[2,2,6,6-tetramethylpiperidin-1-yl]oxidanyl (4-hydroxy-TEMPO or TEMPO) and derivatives thereof. Additional pharmaceutical agents suitable to delivery to an organ according to the disclosures herein include, but are not limited to, metformin, salsalate, oxypurinol, cincalcet, GLPG2737, benzbromarone, niclosamide, senicapoc, pioglitazone, 2-deoxy-D-glucose, a SGLT2 inhibitor, pemafibrate, bardoxolone, sulforaphane, probucol, emalipretide, valproic acid, nicotinamide, and tolvaptan.

The nanoparticles disclosed herein are a drug delivery platform for the purpose of achieving a controlled release profile over time. The nanoparticles comprise one or more pharmaceutical agents disclosed herein dispersed within a polymer matrix, typically a biodegradable, bioerodible, and/or bioresorbable polymer matrix. As used herein, the term “polymer” refers to synthetic homo- or copolymers, naturally occurring homo- or copolymers, as well as synthetic modifications or derivatives thereof having a linear, branched or star structure. Copolymers can be arranged in any form, such as, e.g., random, block, segmented, tapered blocks, graft, or triblock. Polymers are generally condensation polymers. Polymers can be further modified to enhance their mechanical or degradation properties by introducing cross-linking agents or changing the hydrophobicity of the side residues. If crosslinked, polymers are usually less than 5% crosslinked, usually less than 1% crosslinked.

Suitable polymers include, without limitation, alginates, aliphatic polyesters, polyalkylene oxalates, polyamides, polyamidoesters, polyanhydrides, polycarbonates, polyesters, polyethylene glycol, polyhydroxyaliphatic carboxylic acids, polyorthoesters, polyoxaesters, polypeptides, polyphosphazenes, polysaccharides, and polyurethanes. The polymer usually comprises at least about 10% (w/w), at least about 20% (w/w), at least about 30% (w/w), at least about 40% (w/w), at least about 50% (w/w), at least about 60% (w/w), at least about 70% (w/w), at least about 80% (w/w), or at least about 90% (w/w) of the nanoparticles. Examples of biodegradable, bioerodible, and/or bioresorbable polymers and methods useful to make a drug delivery platform are described in, e.g., U.S. Pat. Nos. 4,756,911; 5,378,475; 7,048,946; U.S. Patent Publication 2005/0181017; U.S. Patent Publication 2005/0244464; U.S. Patent Publication 2011/0008437; each of which is incorporated by reference in its entirety.

In aspects of this embodiment, a polymer composing the matrix of a nanoparticle is a polypeptide such as, e.g., silk fibroin, keratin, or collagen. In other aspects of this embodiment, a polymer composing the matrix is a polysaccharide such as, e.g., cellulose, agarose, elastin, chitosan, chitin, or a glycosaminoglycan like chondroitin sulfate, dermatan sulfate, keratan sulfate, or hyaluronic acid. In yet other aspects of this embodiment, a polymer composing the matrix is a polyester such as, e.g., D-lactic acid, L-lactic acid, racemic lactic acid, glycolic acid, caprolactone, and combinations thereof.

One of ordinary skill in the art appreciates that the selection of a suitable polymer for forming the suitable disclosed nanoparticles depends on several factors. The more relevant factors in the selection of the appropriate polymer(s), include, without limitation, compatibility of polymer with pharmaceutical agent, desired release kinetics of pharmaceutical agent, desired biodegradation kinetics at implantation or injection site, desired bioerodible kinetics at implantation or injection site, desired bioresorbable kinetics at implantation or injection site, in vivo mechanical performance, processing temperatures, biocompatibility, and patient tolerance. Other relevant factors that, to some extent, dictate the in vitro and in vivo behavior of the polymer include the chemical composition, spatial distribution of the constituents, the molecular weight of the polymer and the degree of crystallinity.

The disclosed nanoparticles are a drug delivery platform which may include both a sustained release drug delivery platform and an extended release drug delivery platform. As used herein, the term “sustained release” refers to the release of pharmaceutical agents from the disclosed nanoparticles over a period of about seven days or more. As used herein, the term “extended release” refers to the release of pharmaceutical agents from the disclosed nanoparticles over a period of time of less than about seven days.

In aspects of this embodiment, a sustained release drug delivery platform comprising the disclosed nanoparticles releases the one or more pharmaceutical agent with substantially first order release kinetics over a period of, e.g., about 7 days after administration, about 15 days after administration, about 30 days after administration, about 45 days after administration, about 60 days after administration, about 75 days after administration, or about 90 days after administration. In other aspects of this embodiment, a sustained release drug delivery platform comprising the nanoparticles disclosed herein releases the pharmaceutical agents disclosed herein with substantially first order release kinetics over a period of, e.g., at least 7 days after administration, at least 15 days after administration, at least 30 days after administration, at least 45 days after administration, at least 60 days after administration, at least 75 days after administration, or at least 90 days after administration.

In aspects of this embodiment, an extended release drug delivery platform comprising the disclosed nanoparticles releases the one or more pharmaceutical agent disclosed herein with substantially first order release kinetics over a period of, e.g., about 1 day after administration, about 2 days after administration, about 3 days after administration, about 4 days after administration, about 5 days after administration, or about 6 days after administration. In other aspects of this embodiment, an extended release drug delivery platform comprising the nanoparticles disclosed herein releases the pharmaceutical agent disclosed herein with substantially first order release kinetics over a period of, e.g., at most 1 day after administration, at most 2 days after administration, at most 3 days after administration, at most 4 days after administration, at most 5 days after administration, or at most 6 days after administration.

In some embodiments, the nanoparticles are formed of a PLGA-PEG polymer. In some embodiments, the pharmaceutical agents are conjugated to folate before incorporation in to polymer nanoparticle. Conjugation of pharmaceutical agents to folate can be performed by methods known to persons of ordinary skill in the art. Nanoparticles according to the present disclosure are formulated by methods known to persons of ordinary skill in the art. In some embodiments, the nanoparticles have a diameter of about 100-300 nm, about 125-275 nm, about 150-250 nm, or about 175-225 nm.

The disclosed nanoparticles are useful for treating a variety of diseases and disorders. In some embodiments, the disease is a cystic disease. In some embodiments, the disease is a kidney disease. In some embodiments, the kidney disease is polycystic kidney disease. In some embodiments, the disease is a cancer. In some embodiments, the disease is a liver disease. In some embodiments, the disease is a polycystic liver disease.

In some embodiments, the disease is a cystic disease. In some embodiments the cystic disease is a polycystic disease.

In some embodiments, the disease is a liver disease. In some embodiments, the disease is a polycystic liver disease.

In some embodiments, the disease is an ovary disease. In some embodiments, the disease is a polycystic ovary disease.

In some embodiments, the disease is a pancreatic disease. In some embodiments, the disease is a pancreatic cyst disease.

In some embodiments, the disease is a pulmonary disease. In some embodiments, the disease is a pulmonary cyst disease.

In some embodiments, the disease is a vascular disease. In some embodiments, the disease is a peripheral artery disease. In some embodiments, the disease is a vascular aneurysm disease.

Although rapamycin is a very effective drug for treating rodents with polycystic kidney disease (PKD), results in human clinical trials have not been encouraging due to the suboptimal dosages constrained by the side effects. Disclosed herein is the generation, characterization, specificity, functionality, pharmacokinetic, pharmacodynamic and toxicology profiles of novel polycystic kidney-specific-targeting nanoparticles (NPs). These folate-conjugated NPs can be loaded with multiple drugs, including rapamycin and antioxidant 4-hydroxy-TEMPO. The NPs increase the efficacy, potency, and tolerability of rapamycin resulting in increased survival rate and kidney function by decreasing side effects and updated in other organs. The benefits of the previously used daily injection of rapamycin alone can now be achieved with a weekly injection of rapamycin-loaded NPs. The slow sustained-release of rapamycin by kidney-targeting NPs demonstrates a new era of nanomedicine in treatment for chronic kidney diseases at clinically relevant doses.

Compared to the use of rapamycin-NPs, the two most apparent outcomes are that 1 mg/kg/day rapamycin-alone neither increases survival in the Pkd2 mice nor reduces the impediment in body weight gain. The reduction in body weight has in fact been reported in many independent studies, and it is perhaps the most overlooked side effect of rapamycin use in rodents.

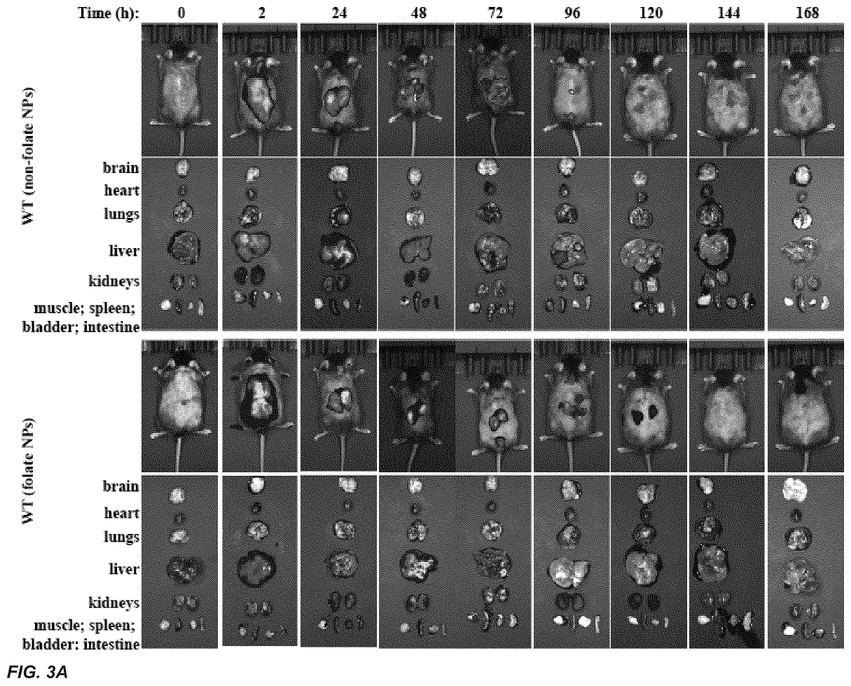

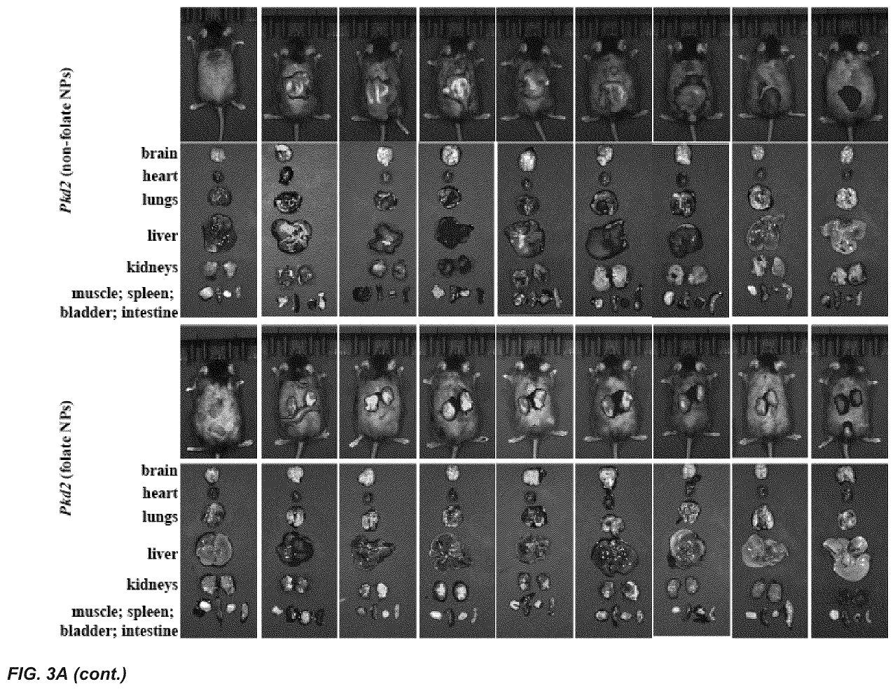

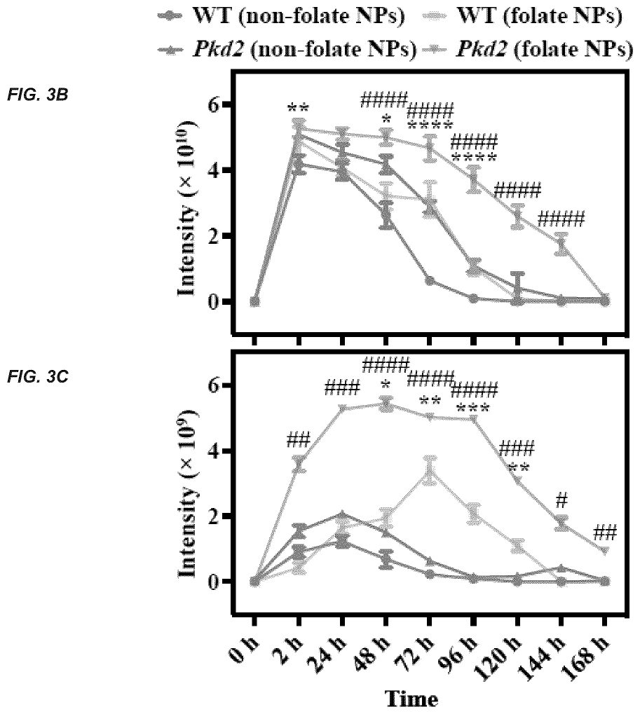

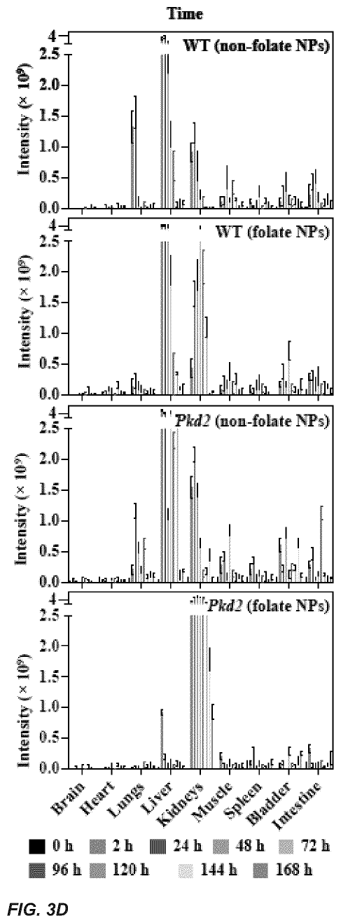

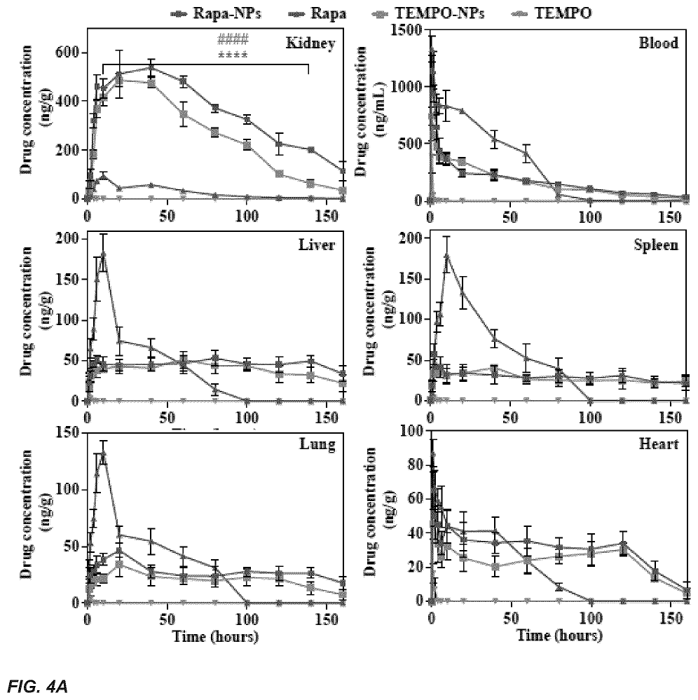

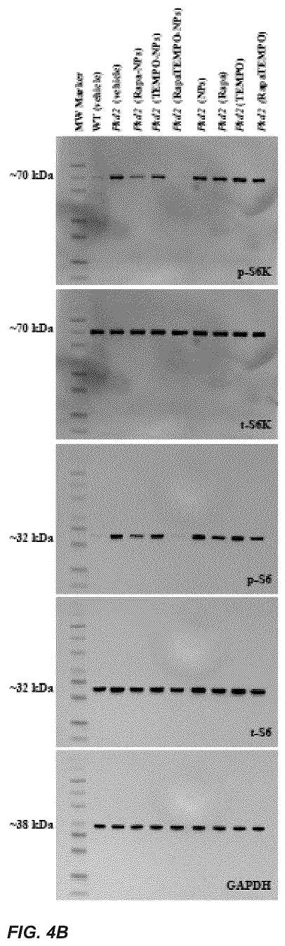

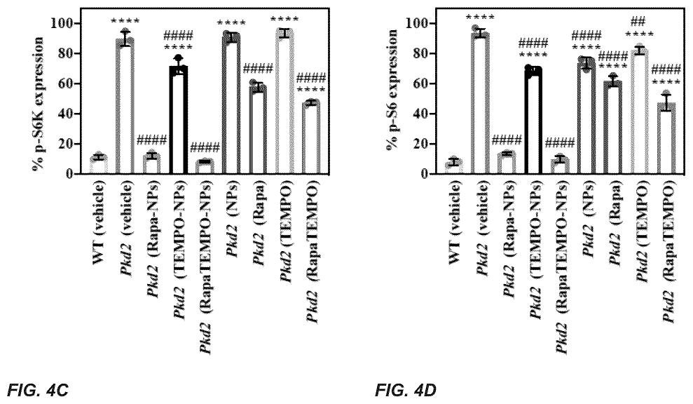

Based on the pharmacokinetic (FIGS. 3 and 4) and pharmacodynamic (FIGS. 4-6 and 18-21) profiles, the administration frequency of rapamycin alone compared to rapamycin-NPs (daily vs. weekly) shows very sub-standard and limited advantages toward PKD. While these advantages of rapamycin alone are beneficial to rodents and, even to some degree, in PKD patients, the sub-standard benefits are masked by the side effects or intolerability. The modest effect of rapamycin is due to the sub-optimal therapeutic dose reaching to the kidney.

PKD is a ciliopathy, and it is thought that primary cilia regulate mTOR signaling pathway. Fluid shear-induced cilia activation through tumor suppressor protein liver kinase B1 can repress mTOR signaling, which in turn regulate cell size and proliferation. The idea of PKD and ciliopathy is further reinforced by a study showing cilia knockout mice had a significant increase in mTOR activity. The present study focused on the downstream of cilia function. Importantly, while there are many aberrant signaling pathways detected in the kidney specific Pkd2 model, including mitogen-activated protein kinase and extracellular signal-regulated kinase pathways, it was found that alteration in mTOR signaling cab partially rescue mice with Pkd2 and cilia-mutant double knockout.

In summary, it was demonstrated pharmaceutical agent-specific delivery of nanoparticles to polycystic kidney disease is an improved therapeutic modality. This nanomedicine approach significantly improves the outcomes of the pharmaceutical agent. The pharmaceutical agent-loaded nanoparticles demonstrate more superior pharmacokinetic and pharmacodynamic profiles than pharmaceutical agent-alone. Furthermore, this nanotechnology offers a potential multiple-therapy strategy with a capability of loading more than one pharmacological agent.

The following definition of terms is provided as a helpful reference for the reader. The terms used in this patent have specific meanings as they related to the present invention. Every effort has been made to use terms according to their ordinary and common meaning. However, where a discrepancy exists between the common ordinary meaning and the following definitions, these definitions supersede common usage.

Aspects of the methods of the present disclosure include, in part, treatment of a mammal. A mammal includes a human, and a human can be a patient. Other aspects of the present disclosure provide, in part, an individual. An individual includes a mammal and a human, and a human can be a patient.

The nanoparticles disclosed herein are generally administered to an individual as a pharmaceutical composition. Pharmaceutical compositions may be prepared by combining the nanoparticles disclosed herein, as an active ingredient, with conventional acceptable pharmaceutical excipients, and by preparation of unit dosage forms suitable for therapeutic use. As used herein, the term “pharmaceutical composition” refers to a therapeutically effective concentration of an active compound, such as, e.g., any of the compounds disclosed herein. Preferably, the pharmaceutical composition does not produce an adverse, allergic, or other untoward or unwanted reaction when administered to an individual. A pharmaceutical composition disclosed herein is useful for medical and veterinary applications. A pharmaceutical composition may be administered to an individual alone, or in combination with other supplementary active compounds, agents, drugs or hormones. The pharmaceutical compositions may be manufactured using any of a variety of processes, including, without limitation, conventional mixing, dissolving, granulating, dragee-making, levigating, emulsifying, encapsulating, entrapping, and lyophilizing. The pharmaceutical composition can take any of a variety of forms including, without limitation, a sterile solution, suspension, emulsion, lyophilizate, or any other dosage form suitable for administration.

A pharmaceutical composition produced using the methods disclosed herein is typically a liquid formulation. A formulation disclosed herein can be produced in a manner to form one phase, such as, e.g., an oil or a solid. Alternatively, a formulation disclosed herein can be produced in a manner to form two phase, such as, e.g., an emulsion. A pharmaceutical composition disclosed herein intended for such administration may be prepared according to any method known to the art for the manufacture of pharmaceutical compositions.

Liquid formulations suitable for parenteral injection may comprise physiologically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethyleneglycol (PEG), glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

A pharmaceutical composition disclosed herein can optionally include a pharmaceutically acceptable carrier that facilitates processing of an active compound into pharmaceutically acceptable compositions. As used herein, the term “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. As used herein, the term “pharmacologically acceptable carrier” is synonymous with “pharmacological carrier” and refers to any carrier that has substantially no long term or permanent detrimental effect when administered and encompasses terms such as “pharmacologically acceptable vehicle, stabilizer, diluent, additive, auxiliary, or excipient.” Such a carrier generally is mixed with an active compound or permitted to dilute or enclose the active compound. It is understood that the active compounds can be soluble or can be delivered as a suspension in the desired carrier or diluent. Any of a variety of pharmaceutically acceptable carriers can be used including, without limitation, aqueous media such as, e.g., water, saline, glycine, hyaluronic acid and the like; solid carriers such as, e.g., starch, magnesium stearate, mannitol, sodium saccharin, talcum, cellulose, glucose, sucrose, lactose, trehalose, magnesium carbonate, and the like; solvents; dispersion media; coatings; antibacterial and antifungal agents; isotonic and absorption delaying agents; or any other inactive ingredient. Selection of a pharmacologically acceptable carrier can depend on the mode of administration. Except insofar as any pharmacologically acceptable carrier is incompatible with the active compound, its use in pharmaceutically acceptable compositions is contemplated. Non-limiting examples of specific uses of such pharmaceutical carriers can be found in Pharmaceutical Dosage Forms and Drug Delivery Systems (Howard C. Ansel et al., eds., Lippincott Williams & Wilkins Publishers, 7^{th} ed. 1999); Remington: The Science and Practice of Pharmacy (Alfonso R. Gennaro ed., Lippincott, Williams & Wilkins, 20th ed. 2000); Goodman & Gilman's The Pharmacological Basis of Therapeutics (Joel G. Hardman et al., eds., McGraw-Hill Professional, 10th ed. 2001); and Handbook of Pharmaceutical Excipients (Raymond C. Rowe et al., APhA Publications, 4^{th} edition 2003). These protocols are routine and any modifications are well within the scope of one skilled in the art and from the teaching herein.

A pharmaceutical composition disclosed herein can optionally include, without limitation, other pharmaceutically acceptable components (or pharmaceutical components), including, without limitation, buffers, preservatives, tonicity adjusters, salts, antioxidants, osmolality adjusting agents, physiological substances, pharmacological substances, bulking agents, emulsifying agents, wetting agents, sweetening or flavoring agents, and the like. Various buffers and means for adjusting pH can be used to prepare a pharmaceutical composition disclosed herein, provided that the resulting preparation is pharmaceutically acceptable. Such buffers include, without limitation, acetate buffers, borate buffers, citrate buffers, phosphate buffers, neutral buffered saline, and phosphate buffered saline. It is understood that acids or bases can be used to adjust the pH of a composition as needed. Pharmaceutically acceptable antioxidants include, without limitation, sodium metabisulfite, sodium thiosulfate, acetylcysteine, butylated hydroxyanisole, and butylated hydroxytoluene. Useful preservatives include, without limitation, benzalkonium chloride, chlorobutanol, thimerosal, phenylmercuric acetate, phenylmercuric nitrate, a stabilized oxy chloro composition, such as, e.g., sodium chlorite and chelants, such as, e.g., DTPA or DTPA-bisamide, calcium DTPA, and CaNaDTPA-bisamide. Tonicity adjustors useful in a pharmaceutical composition include, without limitation, salts such as, e.g., sodium chloride, potassium chloride, mannitol or glycerin and other pharmaceutically acceptable tonicity adjustor. The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. It is understood that these and other substances known in the art of pharmacology can be included in a pharmaceutical composition useful in the invention.

Aspects of the present disclosure include, in part, pharmaceutical agent-containing nanoparticles. As used herein, the term “administering” means any delivery mechanism that provides pharmaceutical agent-containing nanoparticles disclosed herein to an individual that potentially results in a clinically, therapeutically, or experimentally beneficial result.

Administration of pharmaceutical agent-containing nanoparticles disclosed herein include a variety of enteral or parenteral approaches including, without limitation, oral administration in any acceptable form, such as, e.g., tablet, liquid, capsule, powder, or the like; topical administration in any acceptable form, such as, e.g., drops, spray, creams, gels or ointments; buccal, nasal, and/or inhalation administration in any acceptable form; rectal administration in any acceptable form; vaginal administration in any acceptable form; intravascular administration in any acceptable form, such as, e.g., intravenous bolus injection, intravenous infusion, intra-arterial bolus injection, intra-arterial infusion and catheter instillation into the vasculature; perk and intra-tissue administration in any acceptable form, such as, e.g., intraperitoneal injection, intramuscular injection, subcutaneous injection, subcutaneous infusion, intraocular injection, retinal injection, or sub-retinal injection or epidural injection; intravesicular administration in any acceptable form, such as, e.g., catheter instillation; and by placement device, such as, e.g., an implant, a stent, a patch, a pellet, a catheter, an osmotic pump, a suppository, a bioerodible delivery system, a non-bioerodible delivery system or another implanted extended or slow release system. An exemplary list of biodegradable polymers and methods of use are described in, e.g., Handbook of Biodegradable Polymers (Abraham J. Domb et al., eds., Overseas Publishers Association, 1997).

Pharmaceutical agent-containing nanoparticles disclosed herein can be administered to a mammal using a variety of routes. Routes of administration suitable for treating a disorder as disclosed herein include both local and systemic administration. Local administration results in significantly more delivery to a specific location as compared to the entire body of the mammal, whereas, systemic administration results in delivery of pharmaceutical agent-containing nanoparticles to essentially the entire body of the individual. The actual route of administration of pharmaceutical agent-containing nanoparticles disclosed herein used can be determined by a person of ordinary skill in the art by taking into account factors, including, without limitation, the type of disorder, the location of the disorder, the cause of the disorder, the severity of the disorder, the duration of treatment desired, the degree of relief desired, the duration of relief desired, the particular pharmaceutical agent used, the rate of excretion of the pharmaceutical agent used, the pharmacodynamics of the pharmaceutical agent used, the nature of the other compounds to be included, the particular route of administration, the particular characteristics, history and risk factors of the individual, such as, e.g., age, weight, general health and the like, the response of the individual to the treatment, or any combination thereof. An effective dosage amount of pharmaceutical agent-containing nanoparticles disclosed herein can thus readily be determined by the person of ordinary skill in the art considering all criteria and utilizing his best judgment on the individual's behalf.

In an embodiment, pharmaceutical agent-containing nanoparticles disclosed herein are administered systemically to a mammal. In another embodiment, pharmaceutical agent-containing nanoparticles disclosed herein is are administered locally to a mammal. In an aspect of this embodiment, pharmaceutical agent-containing nanoparticles disclosed herein are administered to a site of a kidney disorder of a mammal. In another aspect of this embodiment, pharmaceutical agent-containing nanoparticles disclosed herein are administered to the area of a kidney disorder of a mammal.

### EXAMPLES

### Example 1. Folate Conjugated Nanomedicine for Selective Inhibition of mTOR Signaling in Polycystic Kidneys at Clinically Relevant Doses

Materials. N-hydroxysuccinimide (ThermoFisher), folic acid (Parchem), 3,3′-diethylthiadicarbocyanine iodide (Fisher Scientific) were purchased from different companies and all other Poly(D, L-lactide-co-glycolide) (Resomer® RG 503 H, acid terminated, lactide:glycolide 50:50, MW: 24,000-38,000), poly(ethylene glycol)-bis(amine) (MW: 10,000), methylene chloride, N,N′ dicyclohexylcarbodiimide, dimethyl sulfoxide, rapamycin, 4-hydroxy-TEMPO were obtained from Sigma-Aldrich.

Synthesis and characterization of PLGA-PEG-FA. Poly(D, L-lactide-co-glycolide)-Poly(ethylene glycol)-bis(amine)-folate (PLGA-PEG-FA) was synthesized by the previously reported methods with some modifications. Briefly, 2.5 g of poly(D, L-lactide-co-glycolide) (PLGA) was dissolved in 18.75 mL of methylene chloride and the terminal carboxylic group of PLGA was activated by the addition of 258.75 mg N,N′-dicyclohexylcarbodiimide (DCC) and 143.75 mg N-hydroxysuccinimide to the solution under nitrogen perfusion for a period of 20 h at room temperature condition. The resultant reaction mixture was filtered to remove small (MW<1 kDa) compounds from activated PLGA by adding dropwise to an ice-cold ether, precipitating the activated PLGA. Then, PLGA was dried under vacuum using Biotage V-10 and later dissolved in 20 mL of methylene chloride. The resultant solution was added dropwise into 250 mg/5 mL of Poly(ethylene glycol)-bis(amine) (PEG-NH 2) in methylene chloride solution with a molar ratio of 1:10 for activated PLGA-PEG-NH 2. The reaction was continued for 20 h under nitrogen perfusion and the final product (PLGA-PEG-NH 2) was precipitated by adding the ice-cold ether, filtered and vacuum dried as above. For our final step, the folate conjugation to our diblock copolymer (PLGA-PEG-NH 2), 600 mg was dissolved in 6 mL of dimethyl sulfoxide (DMSO) was mixed with 15.6 mg of FA and DCC. The reaction was continued for 8 h at room temperature. The final solution was added to 200 mL of ice-cold methanol, washed 3 times, and filtered using 2-micron sized filter papers. The resultant product was dried under vacuum and dissolved in 100 mL of dichloromethane for removing any unconjugated FA; this was achieved by centrifuging the solution at 20,000 rpm to separate free FA precipitation. Thus, synthesized PLGA-PEG-FA was characterized using ¹H Nuclear Magnetic Resonance (NMR; 400 MHz Bruker NMR spectrometer), XRD (X-ray diffraction), XPS (X-ray photoelectron spectroscopy) and FTIR (Fourier Transform Infrared Spectroscopy) methods.

PLGA-PEG-FA nanoparticles synthesis and characterization. Plain and different drug-loaded nanoparticle synthesis was carried out by the previously reported nanoprecipitation method with some modifications. For the plain nanoparticles (NPs), PLGA-PEG-FA (200 mg) and 10 mg of 3,3′-diethylthiadicarbocyanine iodide (Cy5, 680 fluorescent emission agent) was dissolved in 4 mL of DMSO. The mixture was then added into the dispersing phase (8 mL of water with 200 μL of 10% Pluronic® F-68) dropwise under moderate magnetic stirring (300 rpm, 1 h). The ratio of diffusing phase to dispersing phase was ˜1:2 (v/v). For the synthesis of rapamycin encapsulated nanoparticles (Rapa-NPs), a combination of PLGA-PEG-FA (600 mg) and rapamycin (20 mg) (1:30) was chosen. Also, 30 mg of 3,3′-diethylthiadicarbocyanine iodide (Cy5, 680 fluorescent emission agent) was dissolved in 12 mL of DMSO. The mixture was then added into the dispersing phase (24 mL of water with 600 μL of 10% Pluronic® F-68) dropwise under moderate magnetic stirring (400 rpm, 1 h). The synthesis of 4-hydroxy-TEMPO encapsulated nanoparticles (TEMPO-NPs) was carried out according to the synthesis method of Rapa-NPs. Particles were lyophilized in a 3% sucrose solution for storage at −80° C. Dried particles were suspended in water and the size and surface zeta-potential of synthesized NPs were obtained by Dynamic Light Scattering (DLS) measurements using a Malvern ZETASIZER® (Nano-ZS; ZEN3600).

For both rapamycin and 4-hydoxy-TEMPO encapsulated nanoparticles (RapaTEMPO-NPs) were synthesized by a combination (1000 mg) and rapamycin (20 mg) and 4-hydoxy-TEMPO (20 mg) (polymer to total drug ratio 1:25) was chosen. Also, 50 mg of 3,3′-diethylthiadicarbocyanine iodide (Cy5, 680 fluorescent emission agent) was dissolved in 20 mL of DMSO. The mixture was then added into the dispersing phase (40 mL of water with 1.2 mL of 10% Pluronic F-68) dropwise under moderate magnetic stirring (500 rpm, 1 h). For size and shape measurements, synthesized nanomaterials were examined by SEM using a TESCAN GAIA-3 XMH integrated focused ion beam-FESEM electron microscope. The size and surface zeta-potential of all synthesized NPs were obtained by DLS measurements using a Malvern ZETASIZER®. All samples of lyophilized NPs were subjected to XRD using a Rigaku SmartLab X-ray diffractometer and Cu-Kα (Cu target) radiation at a scanning rate of 1° per min in the region of 2θ=10-90°. XPS spectra of the samples were recorded on a Kratos Analytical AXIS Supra system with a monochromated Al/Ag X-ray source (Al target). Total survey spectra were recorded in a range from 1300 to −5 eV binding energy (dwell time 200 ms, step size 1 eV, 2 sweeps), and all the region scans were conducted with suitable ranges (dwell time 500 ms, step size 0.05 eV and 5 sweeps). The FTIR spectra were recorded using a Bruker ALPHA (Platinum-ATR) spectrometer in the diffuse reflectance mode at a resolution of 4 cm⁻¹. The rapamycin and 4-yydoxy-TEMPO loading efficiencies were quantified by High-Performance Liquid Chromatography (HPLC; SHIMADZU). Rapamycin and 4-hydoxy-TEMPO release were measured by dialyzing 1 mL of each drug-loaded NP solution at a 5 mg/mL concentration in PBS using 3 k MWCO dialysis tubing and subjected to HPLC. A standard plot was prepared under normal conditions with rapamycin and 4-hydoxy-TEMPO concentrations ranging from 12.5 to 200 μg/mL.

Cell culture. We used primary human epithelial cells for our 3D cyst culture and spectral karyotyping studies. Our lab has previously characterized normal kidney (NK) and ADPKD cells with abnormal cilia functions. NK has fully functional primary cilia, while ADPKD is a well-known model for dysfunctional cilia; thus, we used them as control and abnormal cells in our in vitro studies. We used LLCPK1 epithelial cells obtained from the American Type Culture Collection (ATCC) in some experiments. Human cells were cultured in an epithelial growth medium (PromoCell®) supplemented with 15% fetal bovine serum (FBS; HyClone). LLCPK1 cells were supplied with Dulbecco's Modified Eagle Medium (DMEM; Corning Cellgro®) supplemented with 10% FBS and 1% penicillin/streptomycin (Corning Cellgro). All the cell lines were maintained in 5% CO 2 at 37° C. under humidified culture conditions.

In vitro and in vivo toxicology studies. The in vitro toxicity of synthesized plain NPs, Rapa-NPs, TEMPO-NPs and RapaTEMPO-NPs was performed in renal epithelial cells using Annexin-V (FITC)/propidium iodide apoptosis assay (Molecular Probes). Briefly, cells were treated with different concentrations of 5 to 20 μg/mL of different types of nanoparticles for two days. Healthy, apoptotic, and necrotic cells were distinguished by flow cytometry analysis (BD Facsverse® instrument). Representative images of cells were captured using standard fluorescence and DIC confocal imaging.

Additionally, a fluorescence-activated cell sorting analysis was used to assess the percentages of apoptotic and necrotic cells with a BD Facsvers®e flow cytometer. The obtained data were analyzed using BD-FACsuite® software. Representative images of cells were captured using standard fluorescence and DIC imaging (Nikon Confocal Microscope). For the in vivo toxicity measurements, 200 μL of blood samples were collected from different treatment animals (control and different treatment groups). Hematology and biochemistry were performed using blood-cell analyzers at IDEXX BioAnalytics Services, and a biochemical analyzer (VetScan® VS2, Abaxis).

3D cyst culture and live imaging. Human NK and ADPKD cells were cultured as 3D cyst models according to the manufacturer's protocol and instructions. Briefly, the Corning® Matrigel® basement membrane matrix was thawed overnight by submerging the bottle in ice in a 4° C. refrigerator before use. Once the Matrigel® matrix is melted, the bottle was swirled to ensure that the material was dispersed. On Day 0, the Matrigel® matrix was diluted to 5 mg/mL with an ice-cold complete cell culture medium (DMEM with 10% FBS). NK and ADPKD cells (density of 5×10⁶ cells/mL) were mixed and plated in collagen, EGF and stimulated with forskolin (15 μmol L⁻¹) into pre-chilled p-slide 8 well high glass-bottom chamber slides (ibidi, GMBH) and were coated by adding 100 μL of Matrigel matrix (5 mg/mL) into each well evenly with a pre-chilled pipet tip. The chambered slides were incubated at 37° C. in a humidified atmosphere of 5% CO₂ and media (includes forskolin, 10 μmol L⁻¹) were changed every two days. To assess the effects of vehicle (saline) and different plain and drug-loaded nanoparticles (1 mg/mL) were added (10 μL) on day one after stimulation with forskolin and cyst formation were evaluated on day 6. The cyst growth was observed by using Nikon live cell microscope using its DIC and fluorescence capabilities. Different NP targeting capacities to cilia were observed with a Nikon Eclipse Ti inverted microscope. Using ×20 lens, the number of cysts and their sizes (in diameter) in each well was counted and measured using an ocular scale from the Nikon Eclipse Imaging Software. The microscope is also equipped with an incubator to control 5% CO₂ humidity, 37° C. temperature, and sufficient light to provide a suitable environment for the 3D cultures during the experiment.

Animal studies. All animal procedures were approved by the University of California Irvine or Chapman University Animal Care and Use Committee Guidelines. All animal experimentation was conducted as double-blinded operators to disregard biases and critical analyses. All control (KspCre·Pkd2^{WT/WT}) and PKD (KspCre·Pkd2^{flox/flox}) non-inducible mice models were obtained from the Jackson Laboratory and Harvard Medical School. The mice were then injected with saline (vehicle) plain nanoparticles, rapamycin, TEMPO, and different drug-loaded polymeric nanoparticles (0.25 to 1.0 mg/kg body weight in 100 μL of saline) every seven days total period of 9 weeks via tail vein intravenous injections.

Mouse cardiac and kidney functional studies. KspCre·Pkd2^{WT/WT} and KspCre·Pkd2^{flox/flox} mice (vehicle, plain and drug loaded nanoparticles injected with 0.25-1.0 mg/mL; after 1s 1 week of injections) (total 9 weeks period) were studied to blood pressure monitoring by the noninvasive tail-cuff method using a CODA® high-throughput system (Kent Scientific). Blood pressure was measured twice daily for 9 weeks after the initial 3 days of acclimating each mouse to the tail-cuff. All experiments were performed in a double-blind. The functional kidney tests were conducted at the end of the 9 week treatment, including blood urea nitrogen (BUN), creatinine, and plasma nitrate/nitrite. Creatinine and BUN assays were conducted using an Arbor Assays calorimetric Detection Kit. Plasma nitrate/nitrite concentrations were quantified using a Cayman fluorometric assay kit. All experimental conditions and measurements were performed according to the manufacturers' instructions.

Immunoblotting. After the completion of in-life studies, mice treated with the different control, plain and drug-loaded nanoparticles were euthanized, and the kidneys were collected and rinsed with PBS. Approximately 100 mg of tissue samples were placed in RIPA buffer supplemented with Complete Protease Inhibitor (MedChemExpress). Tissues were homogenized using probe sonication (Fisher Scientific, CL-18) for 15 min at 30 kHz using a pulse of 5 S⁻¹ and 45% acoustic power under the ice. Samples were then centrifuged at 20 000 rpm for 15 min, and the supernatants were collected and subjected to protein quantification. The PAGE (polyacrylamide gel electrophoresis) on 8-10% SDS gels was performed, followed by semi-dry transfer to PVDF membranes using a Bio-Rad Trans-Blot (Turbo Transfer) machine and detection using antibodies against p-S6 (1:000), p-S6K (1:1000), t-S6 (1:2000), t-S6K (1:2000) and GAPDH (1:1,000) (Cell Signaling Technology). Blots were scanned with both calorimetries to image molecular markers and chemiluminescence to capture the protein signal intensity using a BioRad Gel-doc imaging system.

In vivo/ex vivo imaging. After the animal preparation, in vivo and ex vivo optical/fluorescence imaging was performed using the IVIS Spectrum CT imaging system (PerkinElmer). After a white light photographic image was acquired, a fluorescent image was obtained using the following parameters and excitation of 640 nm and an emission of 680 nm. The f-stop used for the image acquisition was 2, exposure time was 5 seconds and the field of view used was 13.4 cm. Isolated kidneys were imaged with the IVIS Spectrum Imaging System and a high-definition camera (Sony Exmor HD-CMOS; Sony) to validate the delivery of the fluorescent nanoparticles to the kidneys. All in vivo and ex vivo images were analyzed and measured by using Living Image software (LI 4.7.3 version; PerkinElmer).

A time-zero image of each treatment group before injection of the agents was taken. According to the manufacturer's instructions, the folate receptor expression in murine polycystic kidneys was measured using kidney-specific FolateRSense™ 680 (PerkinElmer).

Histology Studies. Sections of the mouse visceral organs (hearts, kidneys, livers, spleens, and lung)s, were collected and subjected to hematoxylin and eosin (H&E) staining by starting the tissue fixation using 10% formalin. Then, the tissues were dehydrated in buffered formalin, ethanol, and xylene. Finally, the tissues were embedded in liquid paraffin, sectioned (5 μm), and stained with H&E for histological examinations. The pathology slices were observed and imaged using a KEYENCE BZ-X710 microscope. Mouse normal and polycystic kidney sections were stained with Masson's trichrome to detect fibrosis using a Masson's Trichrome Stain Kit (Polysciences, Inc.).

Data Analysis. The Nikon-A1R confocal microscope images were analyzed and reconstructed with Nikon NIS Element for Advanced Research software. DIC and fluorescent images taken with the Nikon Ti-E were analyzed with NIS-Elements (Nikon; version 4.30) software. For better focusing and live 3D culture imaging, the microscope was equipped with an XY-axis motorized flat top inverted stage, Nikon automatic focusing and a custom-designed vibration isolation platform. All the images taken using Nikon inverted microscopes were captured at the highest resolving power allowed by the system. Scale bars are provided in all DIC and fluorescent images to indicate the actual image reduction size.

All quantifiable data are reported as the mean±standard error mean (SEM). The homogeneity of variance was verified within each data set. When a data set was not normally distributed, or a heterogeneous conflict detected, the distributions normalized via log transformation. This approach produced customarily distributed data sets. Statistical analysis was performed using ANOVA (analysis of variance) followed by Tukey's HSD (honestly significant difference) test. In most of our studies, both the control and test groups were run in parallel; therefore, our control and test values represent matched observations.

In some cases, all the test groups (including the corresponding controls) were analyzed with the post hoc test. In all data sets, power analysis was determined from the coefficient variant. Most of our statistical analyses were performed with GraphPad®-Prism 9.0. In some cases, MS-Excel v.16.0 was used for regression analyses. Linear regression was performed to obtain a standard calibration curve and linear equation. Sample sizes were included in figure legends and graphs. * and # symbols represent statistically significant differences at various probability levels (P).

Results

WComprehensive studies were performed to generate, test and evaluate potency, efficacy, pharmacokinetics, pharmacodynamics and side effects of folate-conjugated PLGA-NPs. PLGA or poly(lactic-co-glycolic acid) was selected because this polymer is known for its biodegradability and biocompatibility. Rapamycin and TEMPO were used in hope to enhance drug efficacies and reduce the undesired side effects in alleviating PKD. To achieve therapeutic levels, rapamycin-alone (1 mg/kg) and/or TEMPO-alone (1 mg/kg) were intravenously injected daily, while NPs (1 mg/kg) were intravenously injected weekly for the in vivo studies. Throughout the studies, KspCre·Pkd2^{WT/WT} and KspCre·Pkd2^{flox/flox} mouse models were used and are respectively referred to wild-type and Pkd2 mice henceforth.

Generating and Characterizing Nanoparticles (NPs)

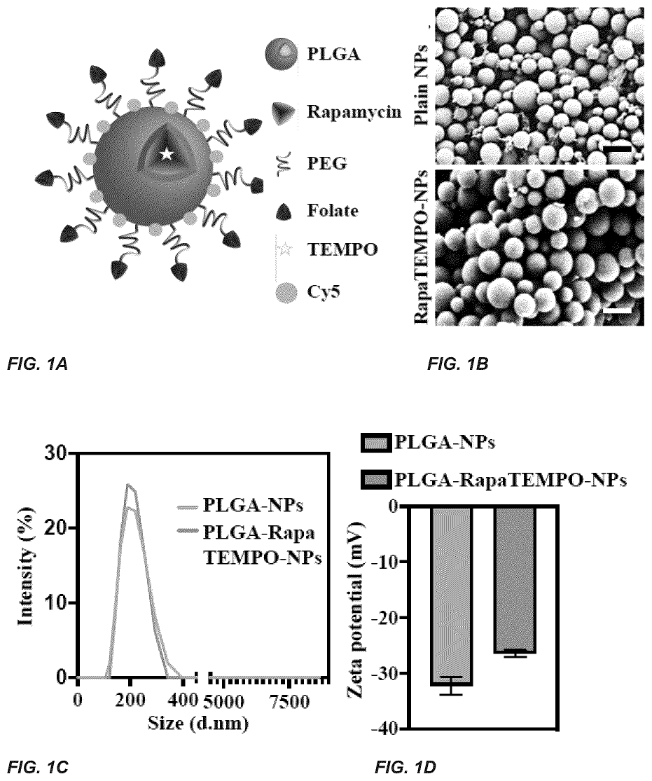

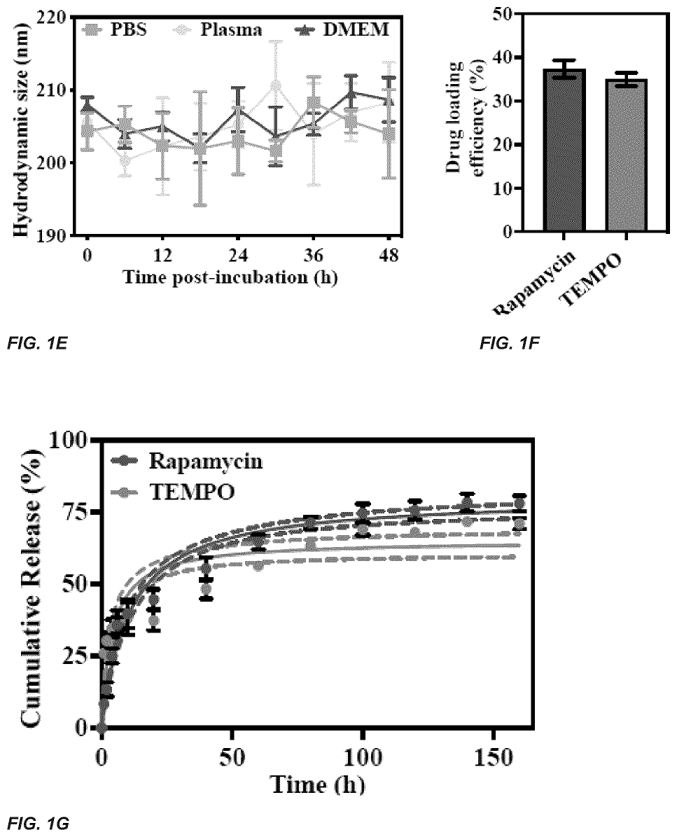

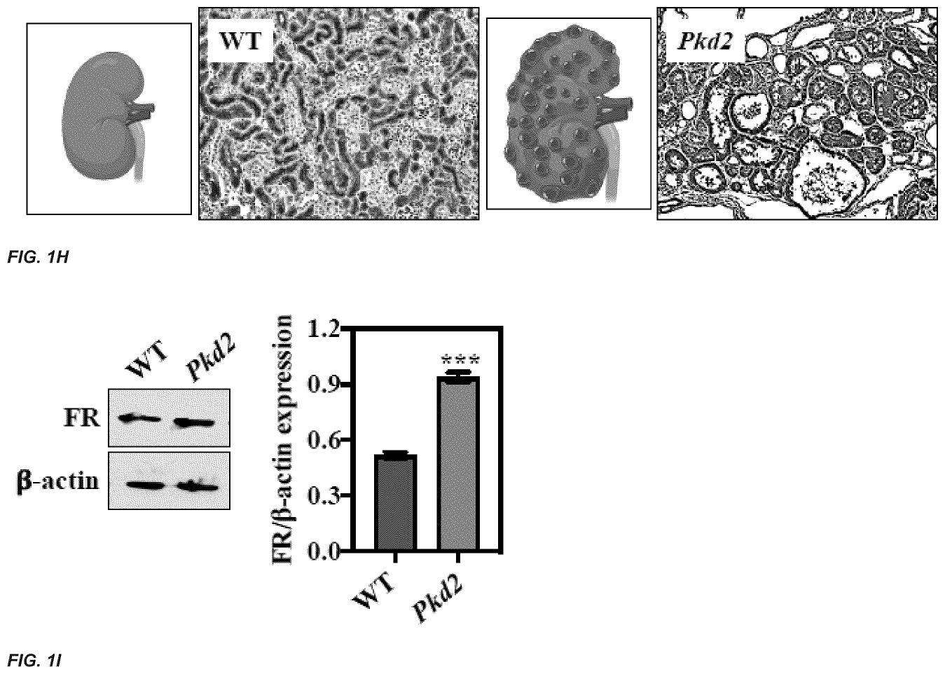

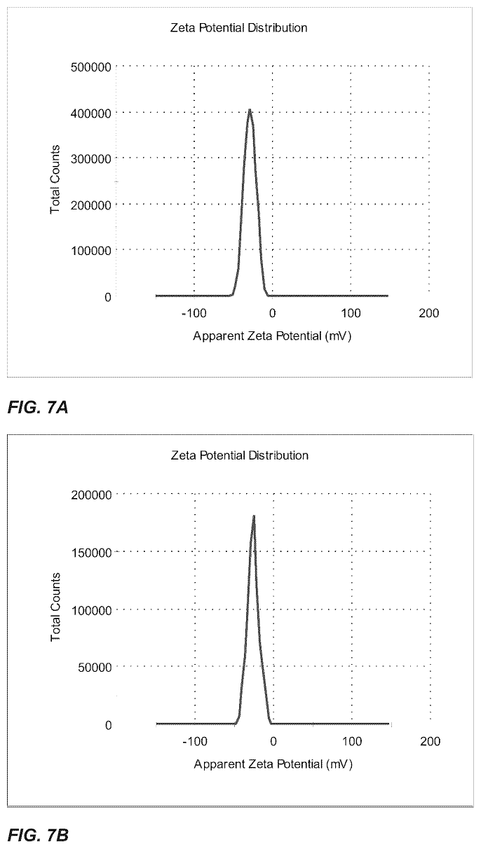

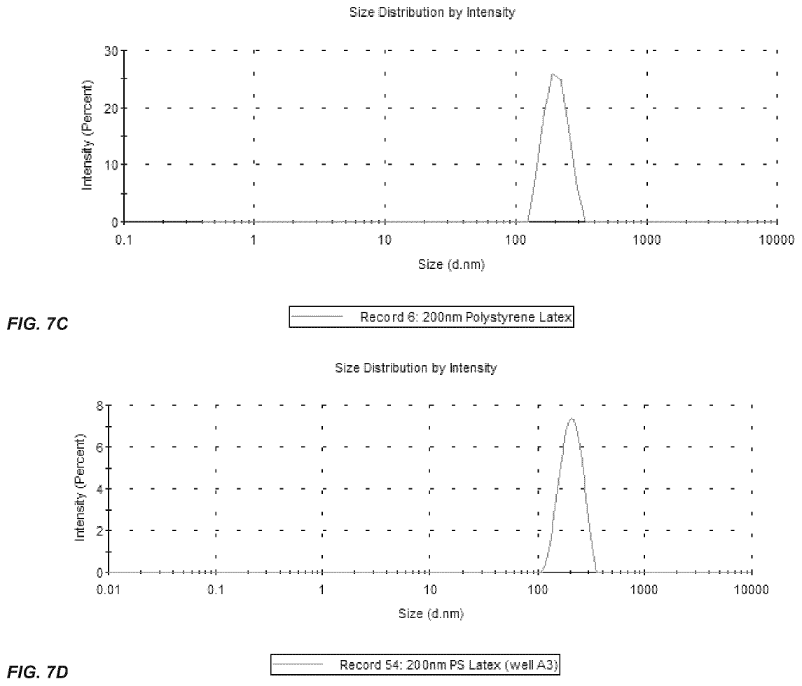

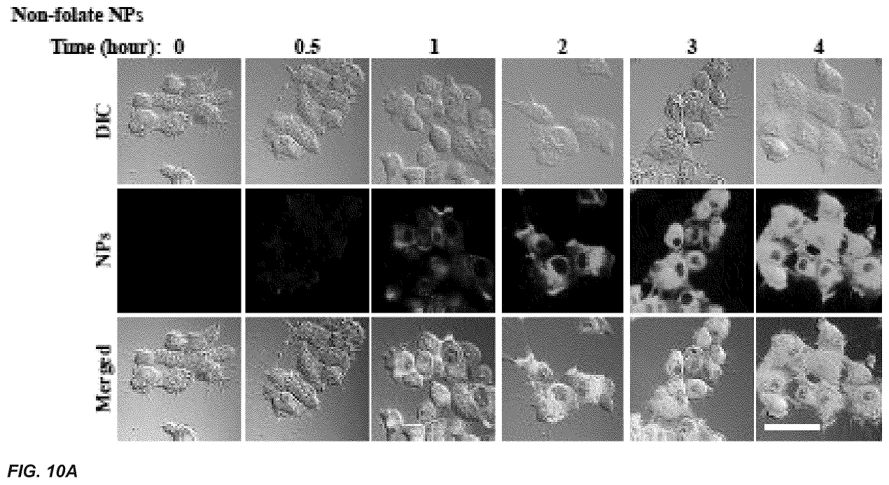

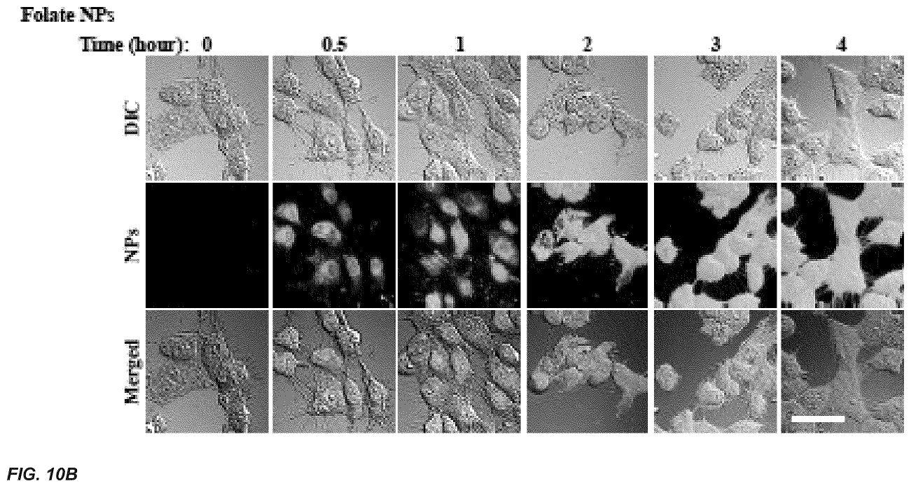

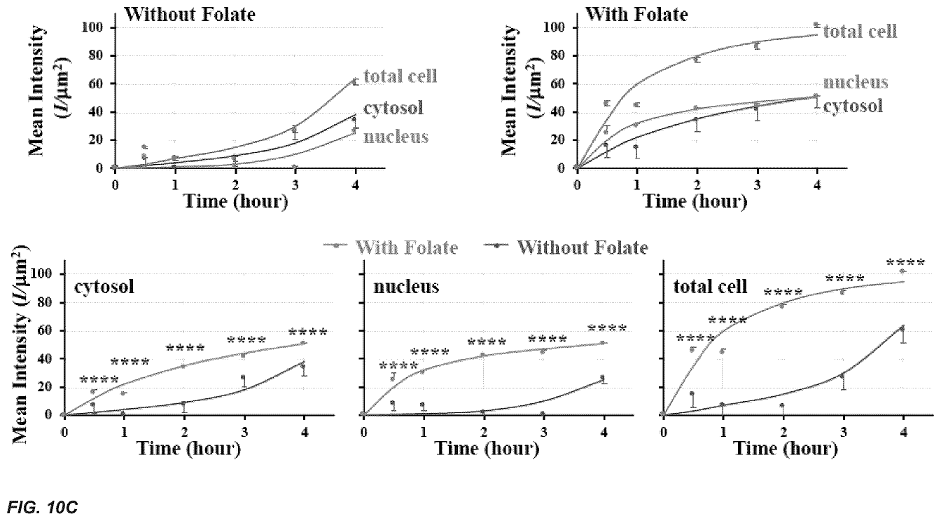

The schematic in FIG. 1 depicts the NP and its interaction among different molecules. The PLGA-based NPs were generated based on the standard synthesis previously disclosed (Pala et al. Nano Lett 19:904-914, 2019). To improve efficiency and avoid pre-mature in vivo degradation, the PLGA-NPs were conjugated with PEG and subsequently folate. Nuclear magnetic resonance structural analyses were performed to validate each synthesis step of PLGA-PEG-folate formation, PLGA alone, PEG alone, PLGA-PEG conjugation, and PLGA-PEG-folate conjugations. The shapes and sizes of the NPs before (202±3.4 nm) and after (214±3.2 nm) drug loading were analyzed with scanning electron microscope (FIG. 1B) and dynamic light scattering (FIG. 10; FIGS. 7A and B). Analysis of surface electrostatic of the NPs before (32.8±3.7 mV) and after (28.2±4.1 mV) drug loading showed an exceptionally high surface negativity (FIG. 1D; FIGS. 7C and D), indicating the dispersion nature of the NPs and the suitability for the in vivo intravenous application.

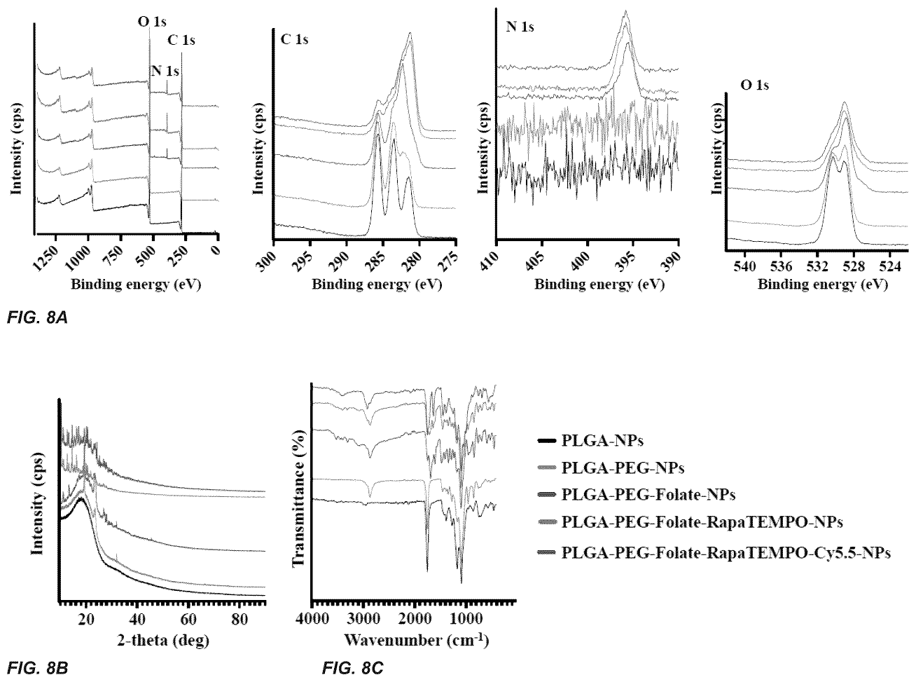

To provide high confidence in the NP synthesis, we evaluated the surface functionalization of the NPs starting from the bare PLGA-NPs to complete functional NPs with PEG and folate before and after the rapamycin-TEMPO loading. In some cases when used for cellular localization and biodistribution studies, the NPs were covalently conjugated with Cy5 mimicking fluorescent dye. The successful formation of each transitional synthesis was characterized, examined and confirmed with X-ray photoelectron spectroscopy with specific analysis of spectra resolutions at C 1s, N 1s and O 1s (FIG. 8A), which was followed by X-ray diffraction spectroscopy (FIG. 8B) and Fourier transform infrared spectroscopy (FIG. 8C). These analyses confirmed the consistency of our synthesis method. They also provided us with the identity the fingerprinting of our functional NPs.

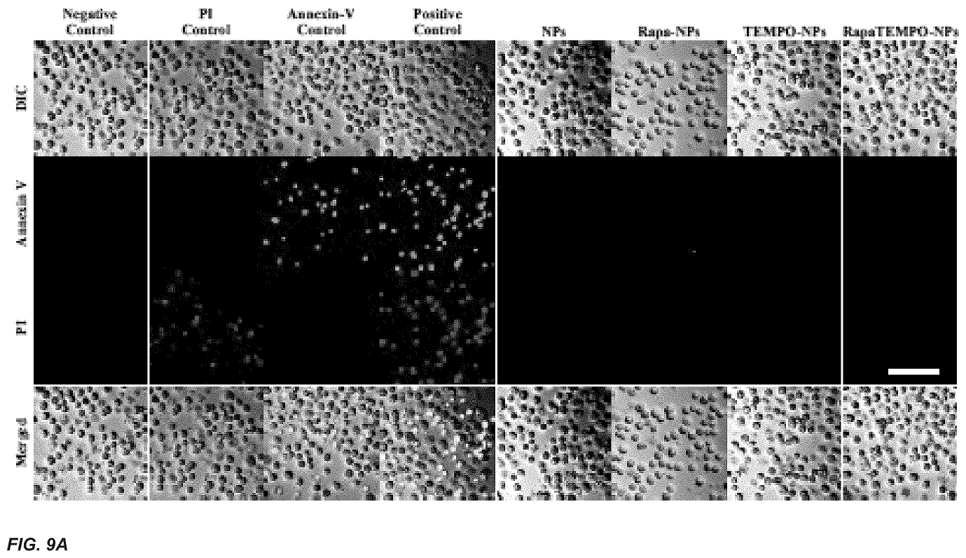

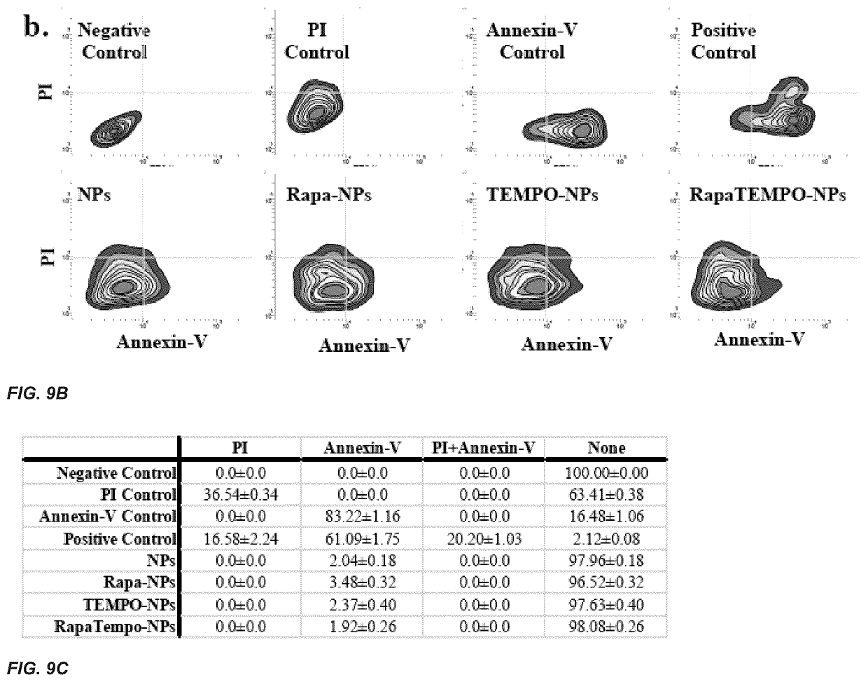

To examine the stability of NPs at room temperature, the NP size was examined in saline, growth media and mouse plasma for 2 days (FIG. 1E). No significant changes in the hydrodynamic size of NPs were observed, indicating the NPs were highly stable. While PEG-based NP formulations are known to have no effect on drug-loading (Peracchia et al. Life Sci 61:749-761, 1997), drug loading in our NPs was further determined by the weight ratios of rapamycin/NPs (37.9±4.2%) and TEMPO/NPs (34.7±3.1%). In addition to drug loading capacity (FIG. 1F), a successful nano-delivery system also depended on the efficiency of drug release (FIG. 1G). Monitoring the drug release over a 7-day period, the efficiencies of rapamycin and TEMPO release were similar with a maximum release at about 3 days. Importantly, neither unloaded nor drug-loaded NPs show in vitro cytotoxicity (FIG. 9). No significant cellular apoptosis or cell death was observed in cells treated for 2 days with NPs-only, rapamycin-loaded NPs, TEMPO-loaded NPs or rapamycin-TEMPO-loaded NPs.

Determining Specificity of NPs In Vitro

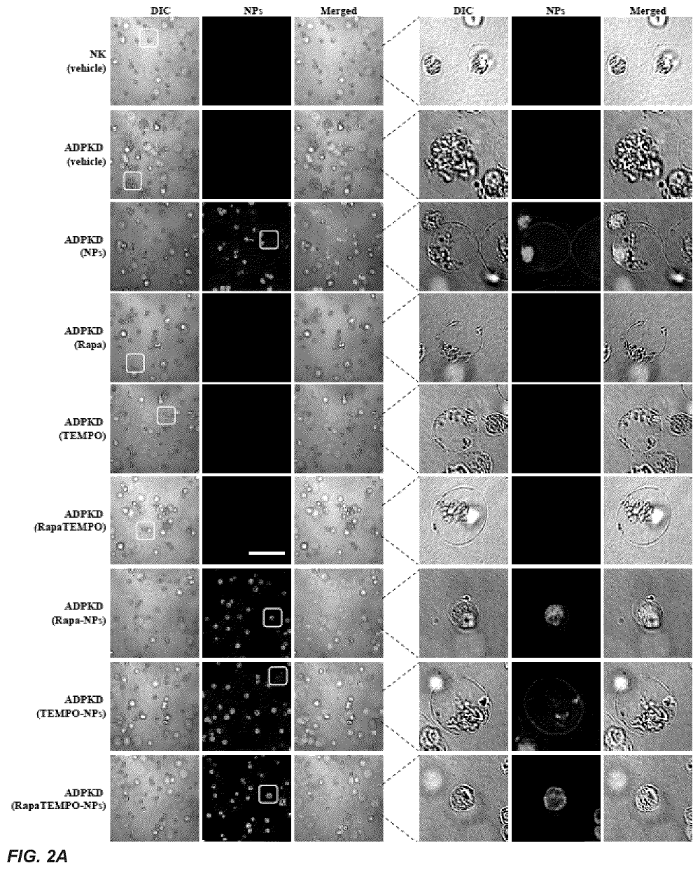

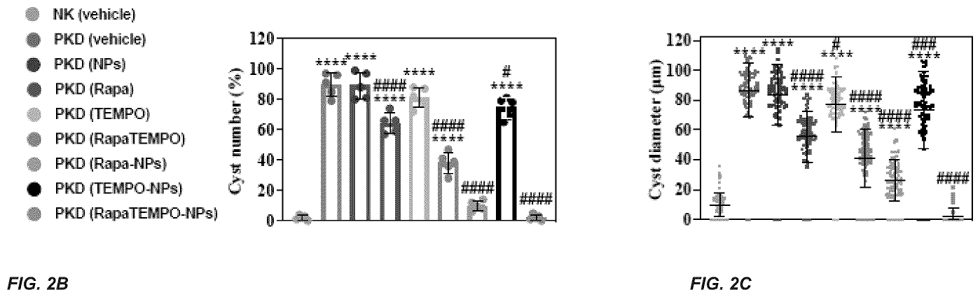

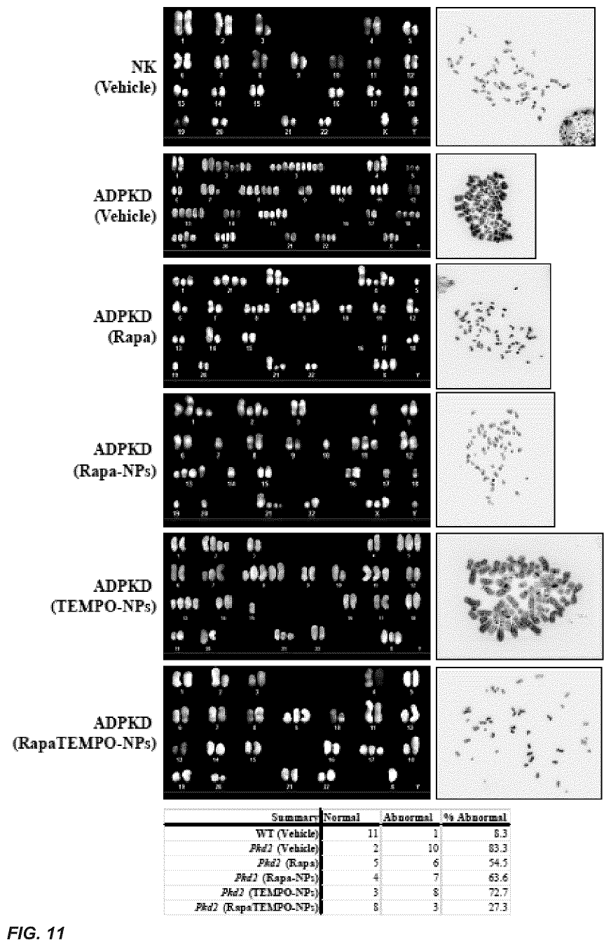

Our strategy of using targeted NPs was based on prior studies showing a higher expression of folate receptors in PKD (Shillingford J M et al. J Am Soc Nephrol 23:1674-1681, 2012; Kipp K R et al. Am J Physiol Renal Physiol 315:F395-F405, 2018). This was also verified in Pkd2 mice with immunohistochemistry and western blot studies (FIGS. 1H and I). The next step was to examine the specificity of our folate-conjugated NPs in cultured cells in vitro (FIG. 10). Compared to unconjugated NPs, folate-conjugated NPs were significantly faster to enter and accumulate in the cells. Cells were eventually overloaded with folate-NPs as evidence from the accumulation of NPs in the nucleus at a significantly faster time and at higher numbers judging from the intensity of fluorescence-labelled NPs. These studies were further validated in a 3-dimension cyst-forming cultured cells in matrigel/collagen matrix, showing that not only the NPs had no observable toxicity, but the rapamycin also significantly decreased the number and size of cysts (FIG. 2). The use of NPs to deliver rapamycin seemed to be more effective than rapamycin-alone, in terms of cyst number and size. While TEMPO-alone or TEMPO-NPs had some effect, the combinational use of rapamycin NPs and TEMPO NPs showed an additive effect. The functional NPs loaded with both rapamycin and TEMPO (referred to as RapaTEMPO-NPs henceforth) showed the most effective treatment among all, as the ADPKD human cells treated with RapaTEMPO-NPs showed a comparable cyst number and size with the normal kidney cells. PKD is associated with polyploidy. Recently, rapamycin was shown to partially correct the abnormal cellular proliferation and polyploidy in PKD and in many types of cancer cells. Thus, karyotyping was performed in ADPKD cells to better identify the effects of RapaTEMPO-NPs on polyploidy (FIG. 11). While rapamycin-alone and rapamycin-NPs did not seem vary much, the use of both rapamycin and TEMPO showed an addictive effect.

Determining Pharmacokinetic Profiles of NPs In Vivo

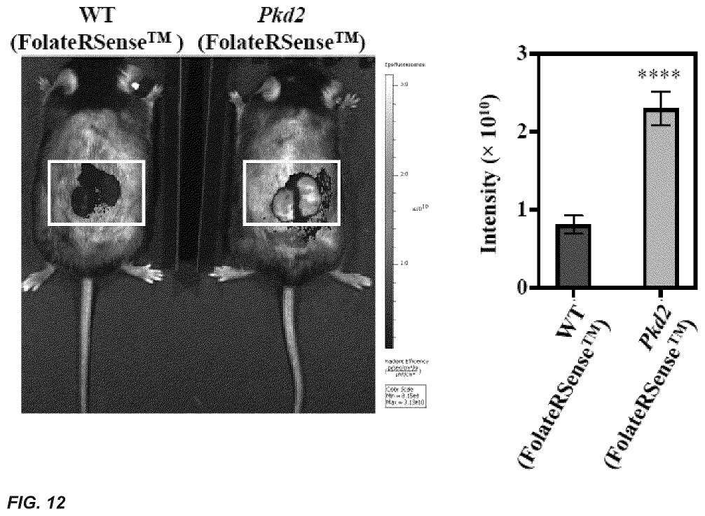

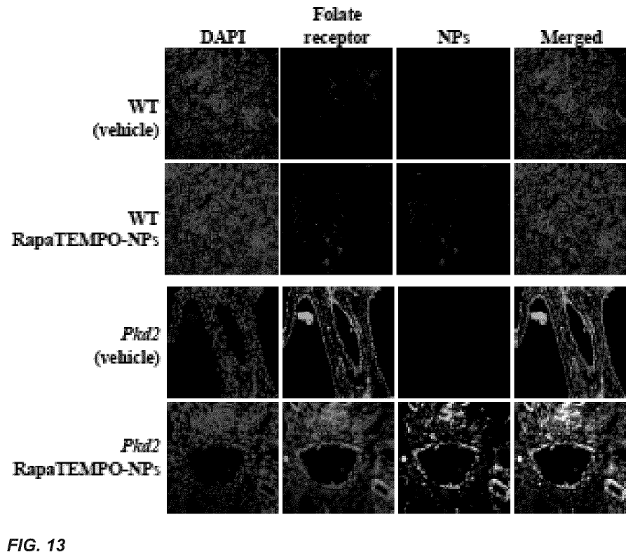

To verify the in vivo specificity of folate-conjugated NPs, analyses were performed using FolateRSense™ 680 (NEV10050; PerkinElmer, Inc; FIG. 12). Commercially available fluorescent-folate allowed us to confirm relative expressions of folate receptors in wild-type and Pkd2 mice 4 hours after injection. Consistent with our data (FIGS. 1H and I), higher expression of folate receptors recruited significantly more fluorescent-folate in Pkd2 than wild-type kidneys. Another study was performed in wild-type and Pkd2 mice at a single time point of 4 hours treatment to ensure that the functional NPs were specifically reaching folate receptors in the kidneys (FIG. 13). This study also validated that Pkd2 kidneys expressed greater folate receptors, resulting in more recruitment of functional NPs.

To understand the pharmacokinetic profile of the non-folate vs. folate-conjugated NPs, additional studies were performed with a single intravenous injection of either NPs for 7 days (FIG. 3A). The specificity of the fluorescence-NP distribution was monitored in a time-dependent manner in wild-type and Pkd2 mice followed with dissection of all visceral organs (brain, heart, lungs, liver, kidneys, muscle tissue, spleen, bladder and intestine). Quantifying the fluorescent-NPs through the whole mice, it was apparent that the peaks of NPs were obtained immediately after injection and subsequently regressed at 2 hours after injection (FIG. 3B). Of particular interest was the Pkd2 mice showing a longer sustained retention of NPs, presumably in the cystic kidneys. This presumption was confirmed after dissection, indicating that the NPs might be distributed in the whole body, but folate-conjugated NPs were significantly accumulated more and faster in the cystic kidneys (FIG. 3C).

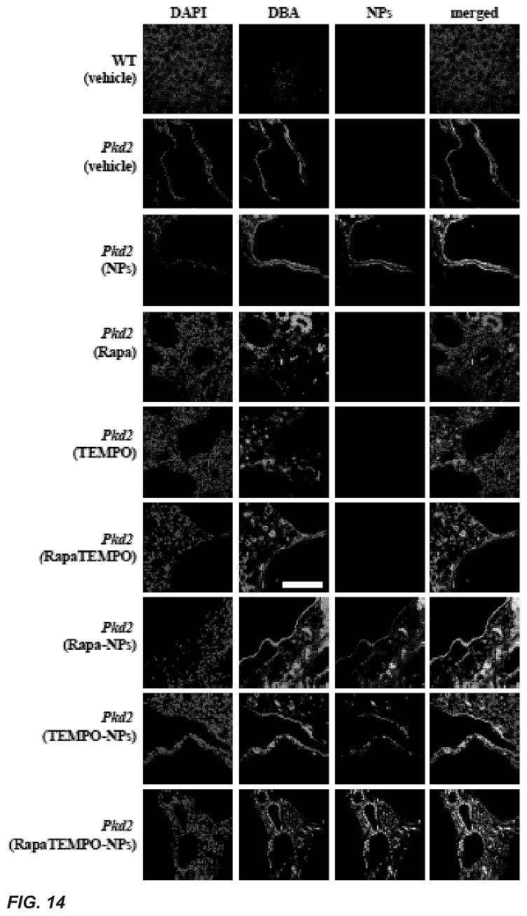

Quantitative analyses of NP distribution was performed (FIG. 3D). The use of NPs with folate-conjugation showed an accumulation and retention of NPs in the targeted kidneys and not in other non-targeted organs; this was more apparent in cystic than normal kidneys. These data also indicated that the pharmacokinetic profiles of the NPs depended on whether the NPs were folate conjugated, and if the kidneys had cysts. Therefore cystic kidney-specific targeting was now available, suggesting that potential different payloads could also be used in the future. To further understand the distribution of NPs in the cystic kidneys, we analyzed the localization of NPs at 24 hours (FIG. 14). While NPs could be confirmed to localize cyst-lining epithelia, they could be found elsewhere, most likely where the folate receptors were expressed (FIG. 1H).

Validating the Pharmacodynamic Profiles of NPs

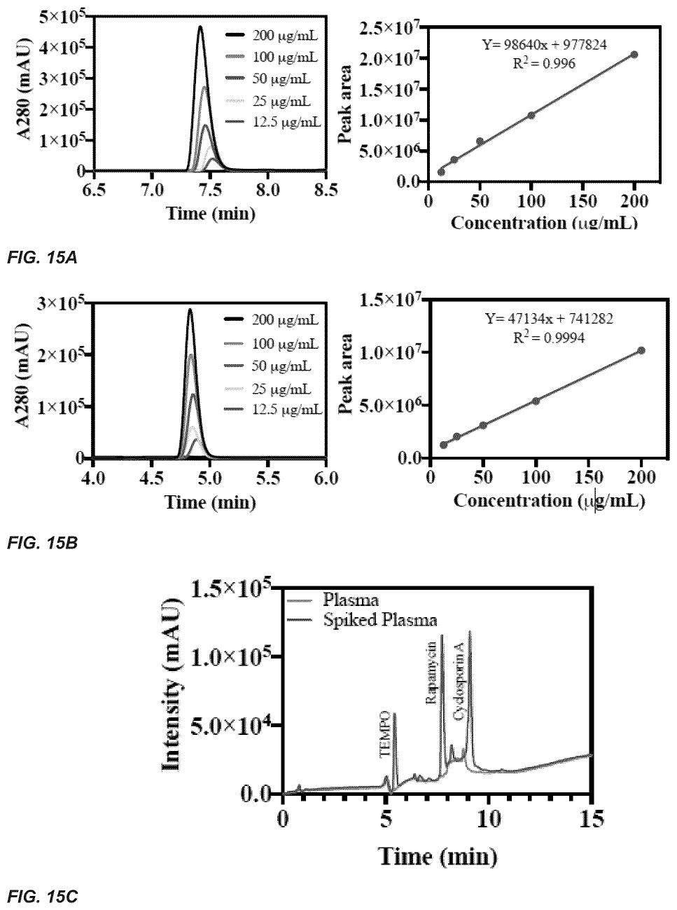

Having NPs as a specific kidney delivery composition did not necessary equate to the functionality of NPs in the kidneys. The next step was therefore to analyze effects of NPs in the kidneys by first examining the release of rapamycin and/or TEMPO from the NPs. Based on the pharmacokinetic profiles, standardized analyses were generated using high-performance liquid chromatography (HPLC) to quantify rapamycin (FIG. 15A) and TEMPO (FIG. 15B); a typical representative HPLC spectra was therefore used to ensure consistent measurements of the drug release and no chemical interaction between rapamycin and TEMPO (FIG. 15C). This standard was used to examine accumulation of drugs (i.e., rapamycin and TEMPO) in the visceral organs (FIG. 4A). The pharmacodynamic profiles of the drug distribution was consistent with the NP distributions (FIG. 3) that 1) NPs kept drugs longer in the body, 2) NPs retained drugs significantly longer and at higher concentrations in the kidneys, and 3) NPs provided a sustained-drug release for a minimum of 7 days. This allowed us to formulate an injection regiment of NPs for every 7-day duration.

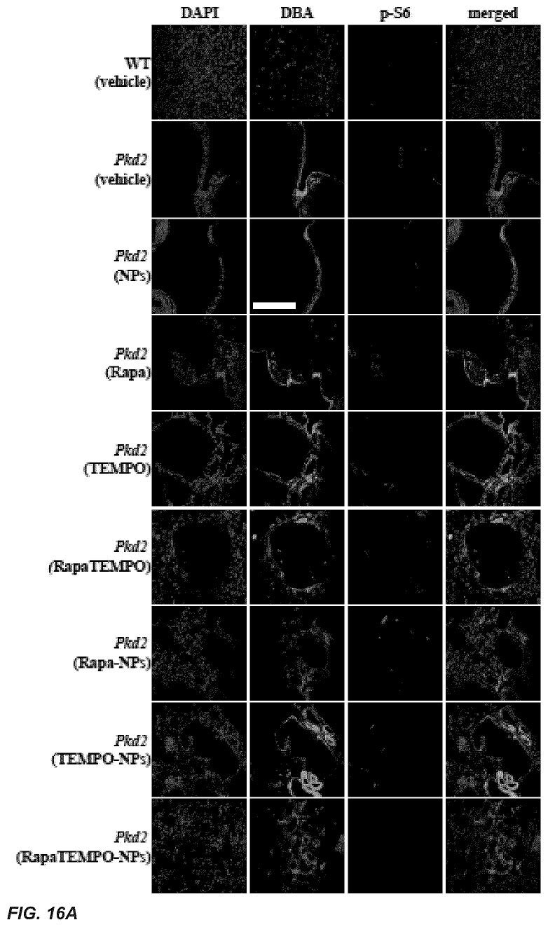

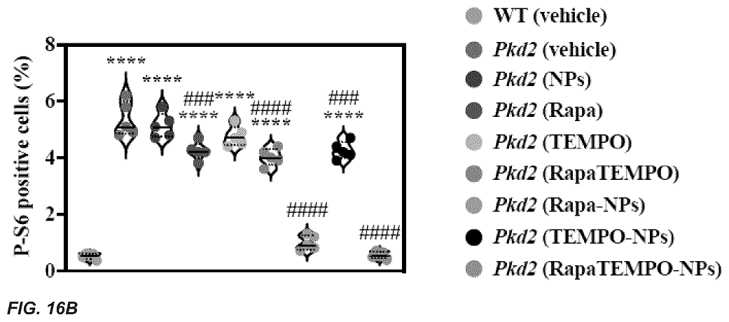

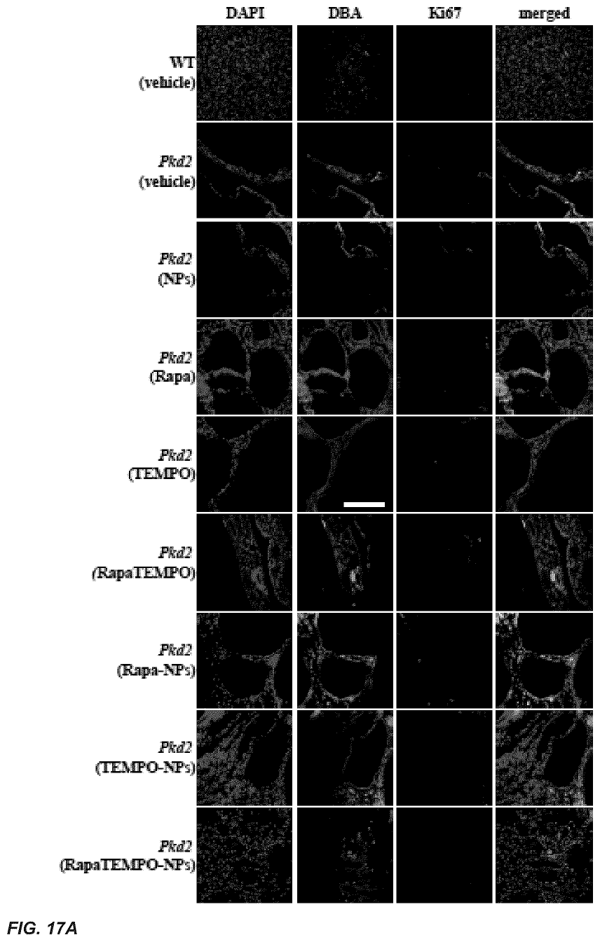

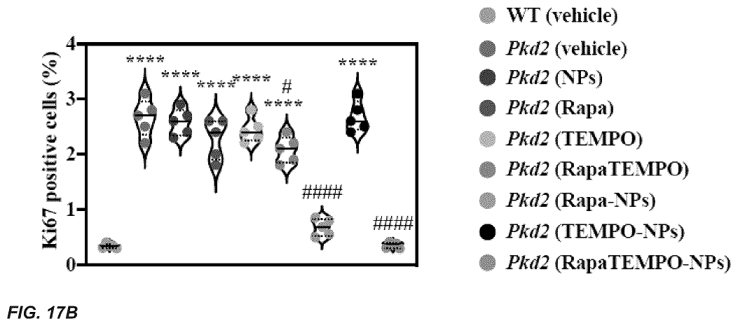

The molecular effects of rapamycin released from NPs were further examine to evaluate the functionality of rapamycin in the kidney at the end of 9 week of therapy period (FIG. 4B). The use of rapamycin in PKD is based primarily on the consensus that the mTOR pathway is activated in cystic kidneys. Upon mTOR inhibition by rapamycin, S6 kinase (S6k) were dephosphorylated and inactivated in cystic kidneys (FIG. 4C). This resulted in the inhibition of the downstream phosphorylation of S6K substrate (S6; FIG. 4D). The contralateral kidneys in these studies were also used to confirm phosphorylated-S6 with immunofluorescent method (FIG. 16). While TEMPO-alone had a minimal effect, TEMPO potentiated the effect of rapamycin. Such a potentiating effect of rapamycin/TEMPO was not seen with the NPs, because NPs were very effective to deliver a targeted effect. While rapamycin and/or TEMPO without NPs showed some significant effects, the effects were significantly amplified as evidence on the levels of phosphorylation of S6K and S6. Importantly, the use of RapaTEMPO-NPs significantly reduced Ki-67 positive cells (FIG. 17), a common proliferation index in cystic kidneys.

Determining the Therapeutic and Side Effects of NPs

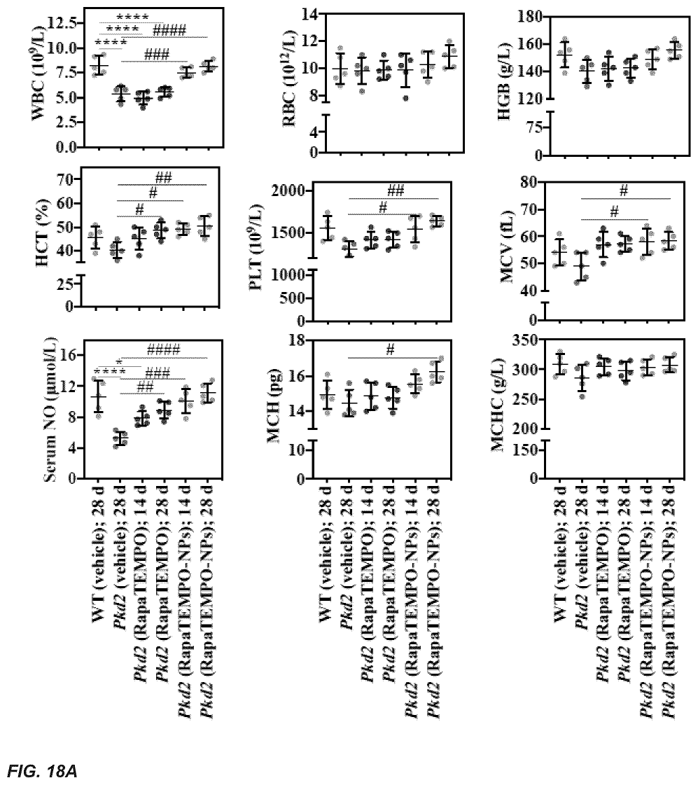

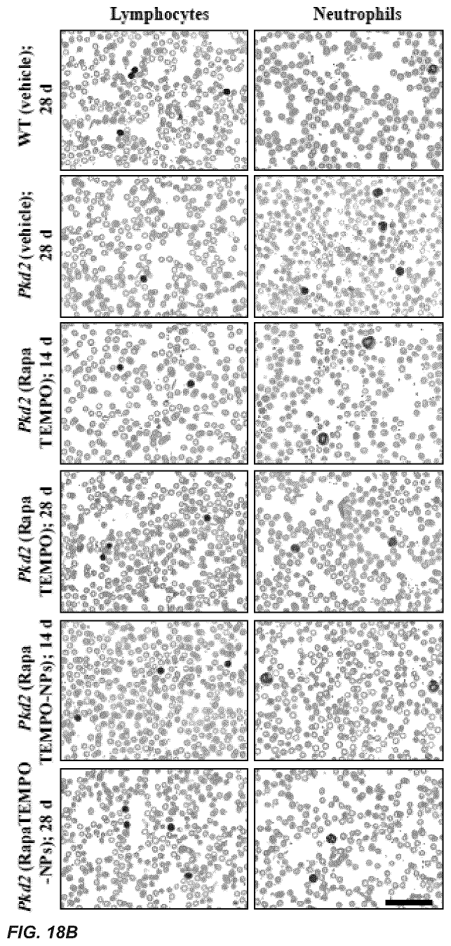

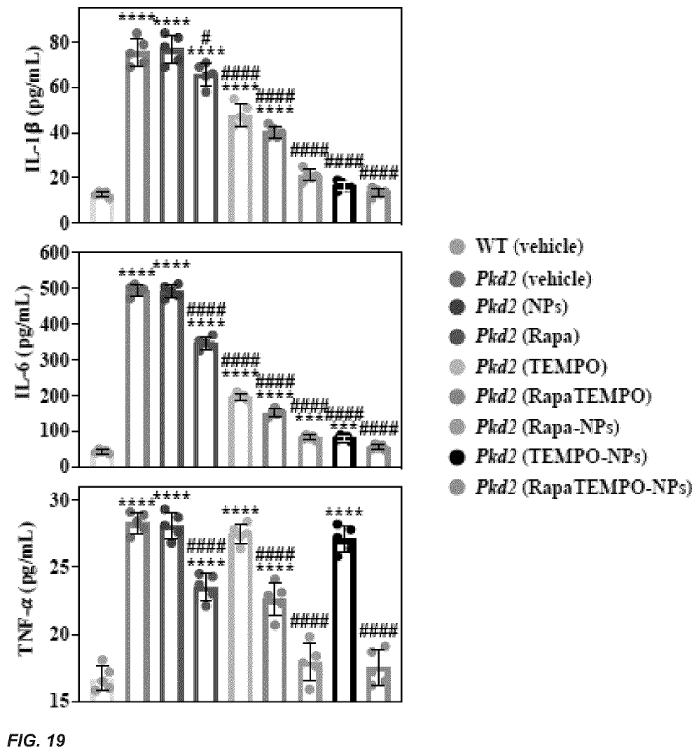

Comprehensive in vivo studies on blood work were performed to look at effects of rapamycin-TEMPO without and with NPs at 14- and 28-days post treatment (FIG. 18). A routine complete blood count test was performed independently by a third-party CRO-services (IDEXX BioAnalytics). The levels of different components of every major cell in the blood were quantified, including white blood cells (WBC), red blood cells (RBC), hemoglobin (HGB), hematocrit (HCT), platelets (PLT), mean corpuscular volume (MCV), serum nitric oxide (NO), mean corpuscular hemoglobin (MCH) and mean corpuscular hemoglobin concentration (MCHC). Because the kidney-specific Pkd2 mouse model was used, the most surprising results were that NO and WBC were significantly lower in Pkd2 than wild-type mice (FIG. 18A). While NO had been used as an indicator of vascular function in PKD, our finding supports the interaction of vascular and renal functions via the Renin-Angiotensin-Aldosterone System (RAAS) that was known to be elevated and subsequently impeding vascular function in PKD. RapaTEMPO-NPs improved serum NO as soon as 14-days, while drugs alone took about 28 days post treatment. The blood smear for lymphocytes and neutrophiles was performed (FIG. 18B), because they were cells known for fighting common infections and likely to be affected by drugs. While the mechanistic link between WBC and PKD is not clear, PKD patients independent of their kidney function are known to have cytopenias and lymphopenia. Importantly, the lower WBC in PKD mice could be rescued with RapaTEMPO-NPs but not drugs alone in as little as 14 days. The key inflammatory cytokines, IL-1E, IL-6 and TNF-□, were measured in the kidneys (FIG. 19). While a significant inflammation in cystic kidneys was not surprising, our data further confirmed that RapaTEMPO-NPs would significantly improve cytokine release to the levels comparable to wild-type control. TRapaTEMPO-NPs showed to significantly improve the blood count and kidney inflammation.

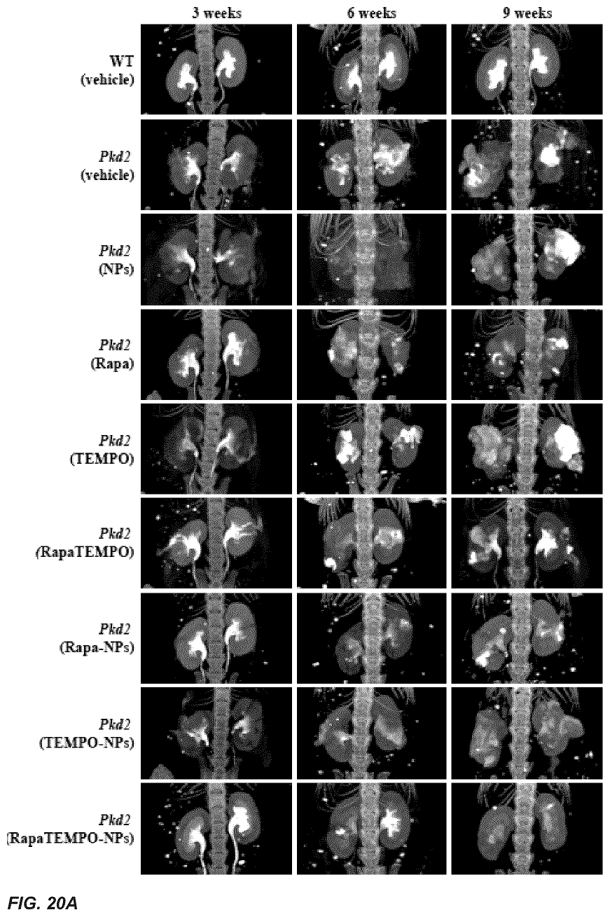

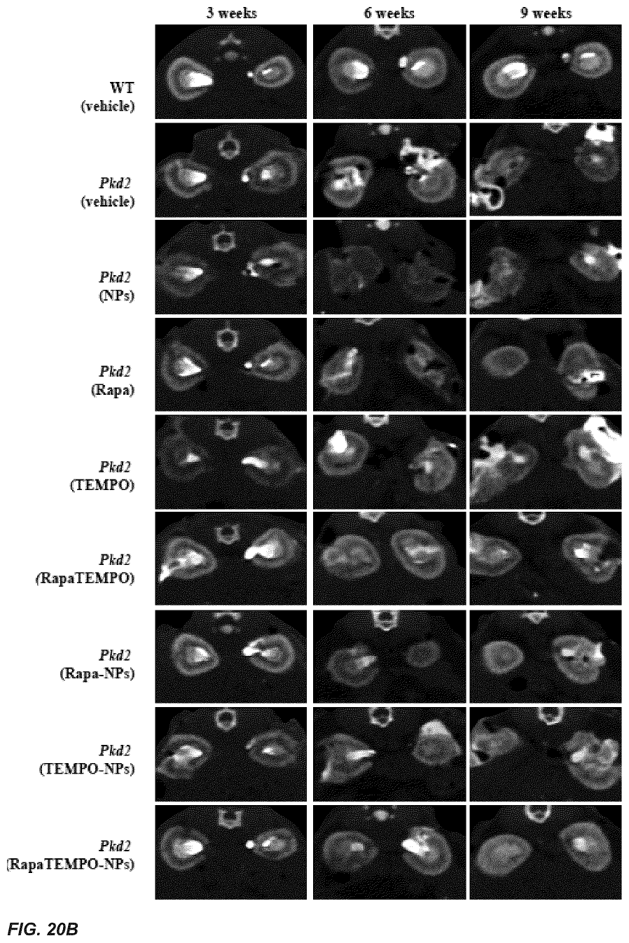

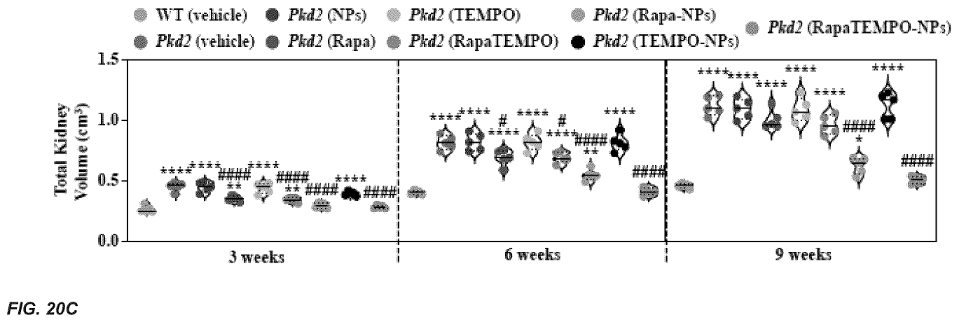

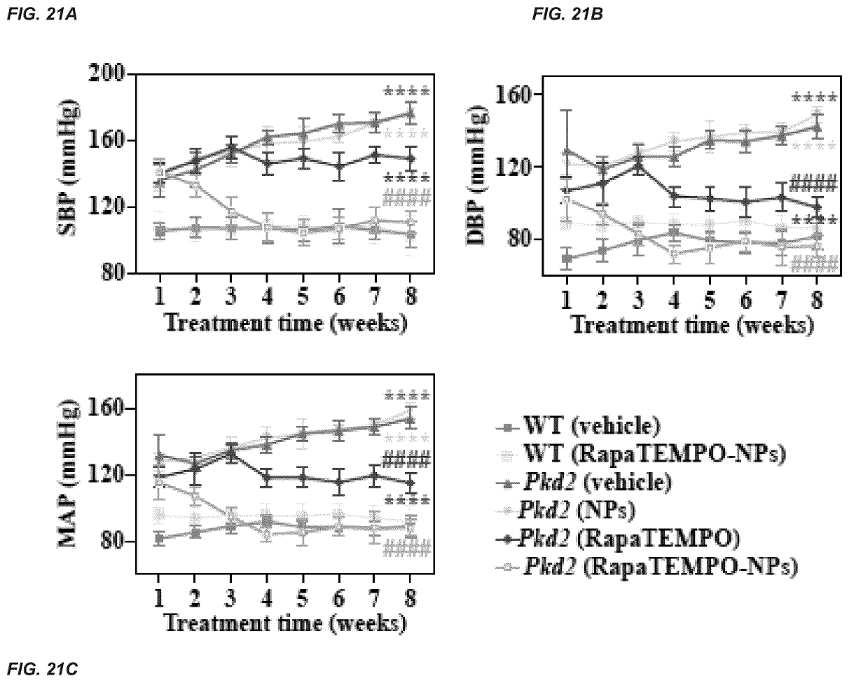

Because RapaTEMPO-NPs were applied prior to severe cystic development in the kidneys, lateral (FIG. 20A) and cross-section (FIG. 20B) of computerized tomography scans were performed in the kidneys at 3-, 6- and 9-weeks to monitor progression of changes in kidney volume. The quantitative kidney volume analyses indicated that rapamycin-alone showed a promising effect at 3- and 6-weeks, while Rapamycin-NPs or RapaTEMPO-NPs exhibited promising results throughout the duration of treatment (FIG. 20C). Compared to Rapa-NPs, RapaTEMPO-NPs significantly repressed total kidney volume to a level comparable to wild-type kidneys. This suggested that while TEMPO showed no effect in kidney volume expansion, the use of TEMPO in RapaTEMPO-NPs demonstrated an additional protective effect in the kidneys. Longitudinal effects on blood pressure were also monitored throughout the treatment period (FIG. 21). The increase in blood pressure in kidney-specific Pkd2 seemed to be associated with expansion of renal cysts leading to compression in renal vasculature and eventual RAAS activation. Importantly, TEMPO potentiated the effect of RapaTEMPO-NPs, which significantly lowered the blood pressure to a level comparable to wild-type mice.

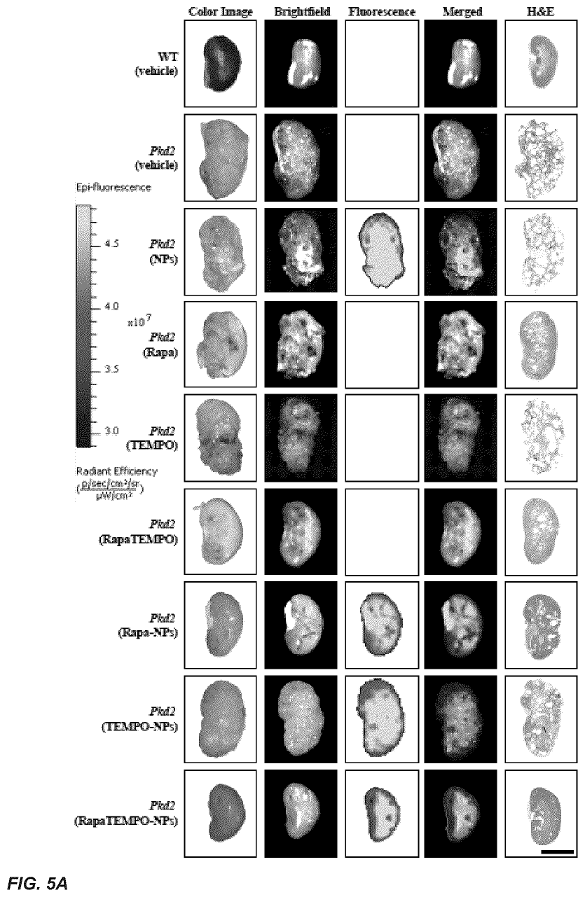

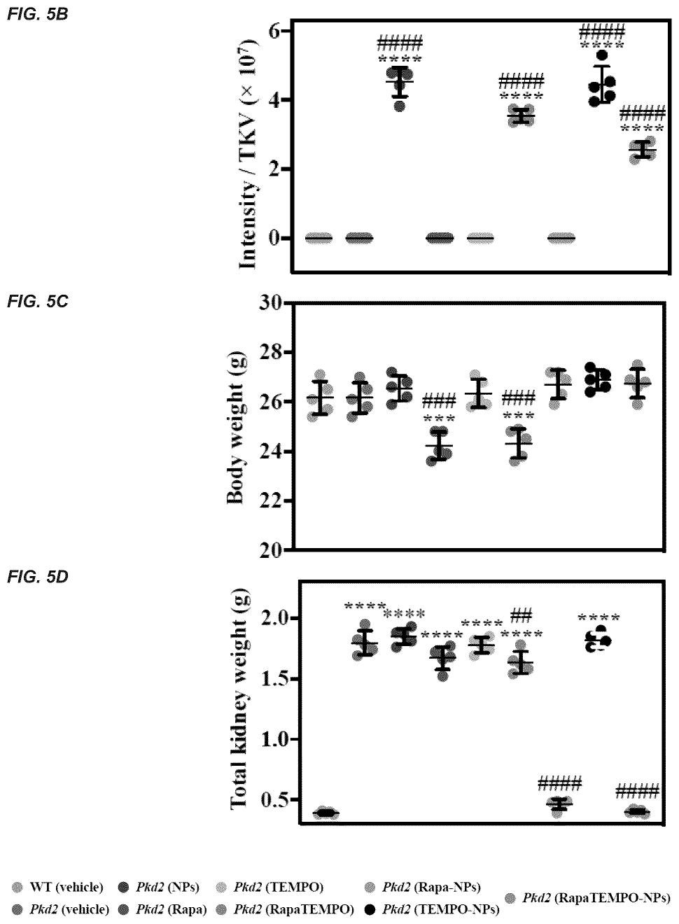

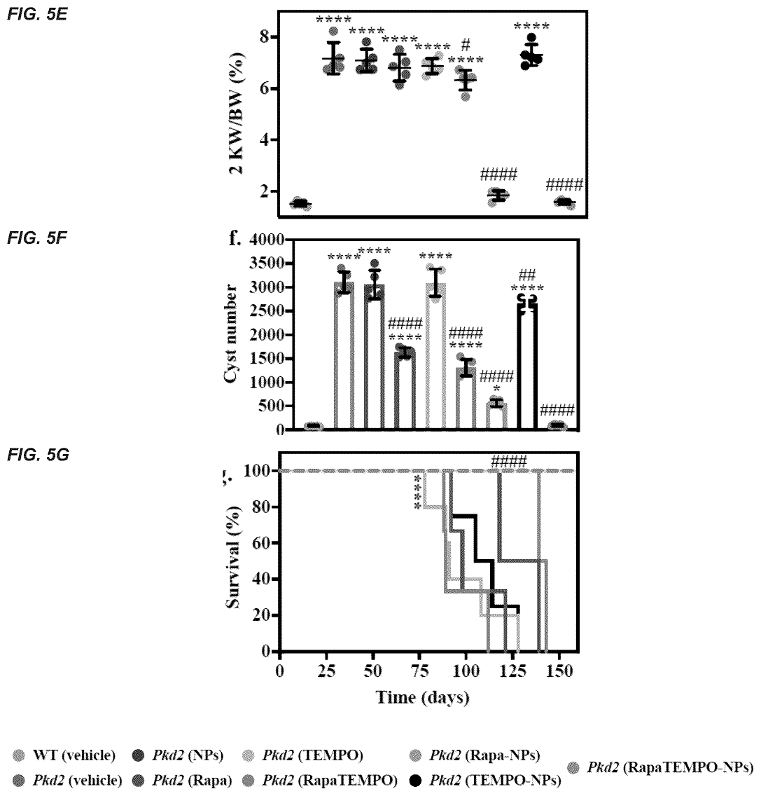

At the end of 9 weeks of treatment, kidneys were isolated, imaged, examined and used for quantitative analyses. The kidney morphologies were imaged for qualitative structural observation, assessed for NP incorporation, and processed for a standard H&E staining (FIG. 5A). The distributions of NPs were again confirming NP specificity to cystic kidneys (FIG. 5b). The accumulation of NPs was associated in kidneys with more severe cysts after normalized the measurement to kidney volume. Compared to control or NPs, the long-term use of rapamycin-alone or rapamycin with TEMPO significantly lowered the body weight (FIG. 5C). This pointed to the potential side effect, intolerable dosage or unreasonable long-term use of rapamycin.

Compared to kidneys from wild-type mice, the polycystic kidneys significantly had greater weights (FIG. 5D) and kidney to body weight ratios (FIG. 5E). Quantitation of cyst number indicated that while rapamycin-alone significantly decreased cyst number compared to Pkd2 vehicle group, RapaTEMPO-NPs significantly prevented cyst formation to a level comparable to wild-type mice (FIG. 5F). This suggested that TEMPO potentiated the effects of Rapa-NPs. Importantly, RapaTEMPO-NPs significantly increased the survival rate (FIG. 5G).

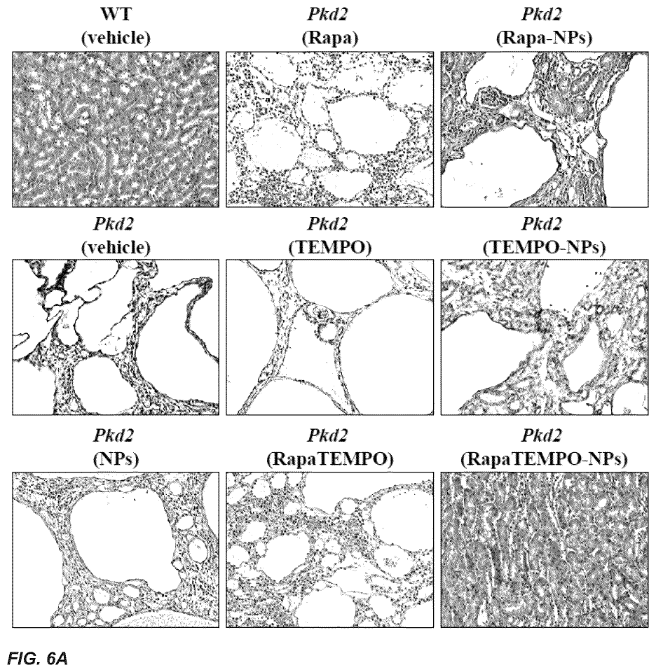

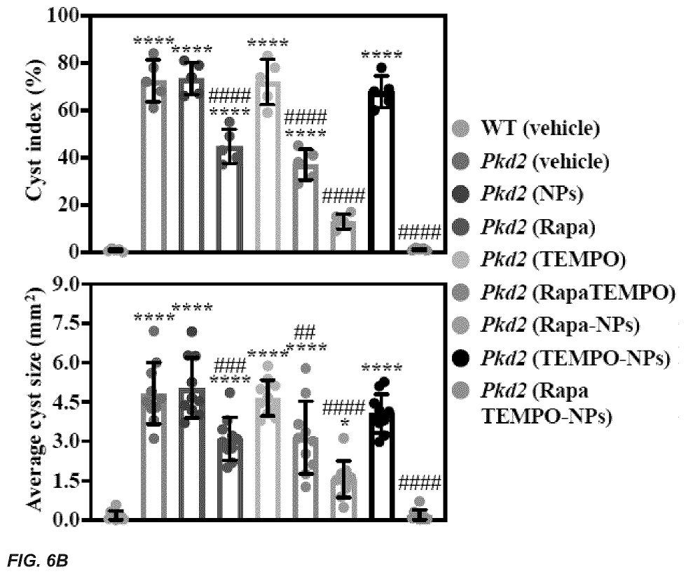

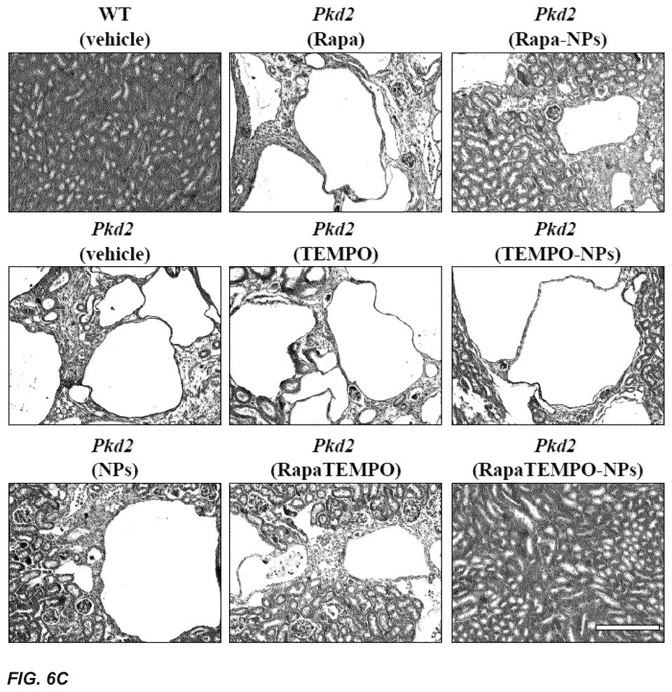

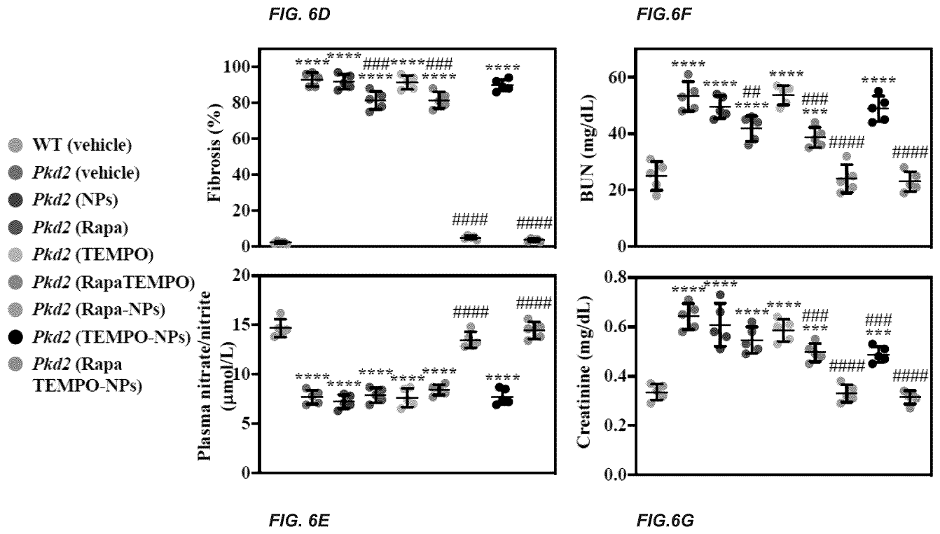

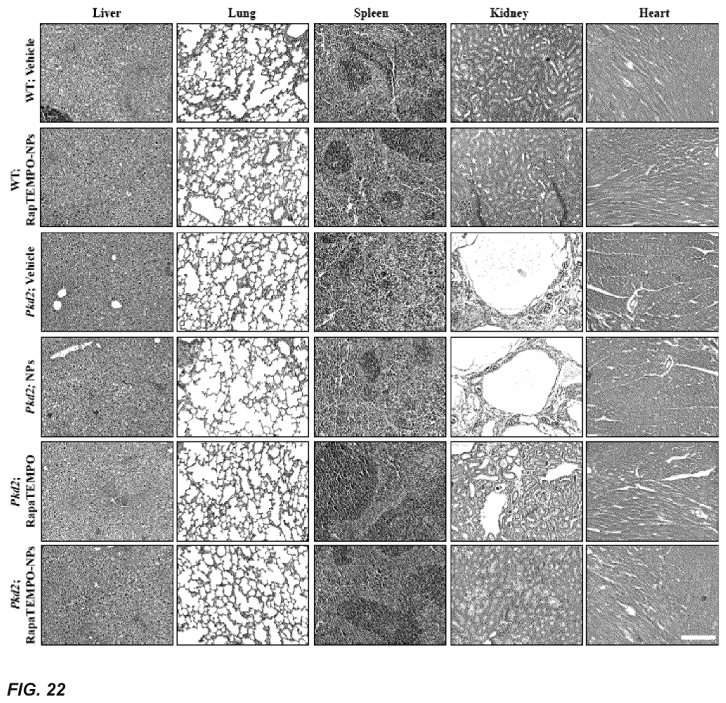

A quantitative analysis was performed on cystic index and size with standard H&E staining (FIGS. 6A and B). Renal fibrosis was also quantified with Masson's trichrome staining (FIGS. 6C and D). Again, RapaTEMPO-NPs demonstrated significant superiority in treatment than rapamycin-alone. TEMPO significantly potentiated effect of RapaTEMPO-NPs on cyst size and played an important role to reduce fibrosis in targeted therapy. In these studies, Rapa-NPs or RapaTEMPO-N Ps were shown to be equally effective to correct plasma nitrate to the level of control wild-type (FIG. 6E). Renal functions were also confirmed to be corrected by either Rapa-NPs or RapaTEMPO-NPs (FIGS. 6F and G). Histopathology was done to confirm that the use of NPs did not result in any organ toxicity (FIG. 22).

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. Nanoparticulate formulations of pharmaceutical agents for the treatment of chronic kidney diseases, the nanoparticles comprising one or more pharmaceutical agents encapsulated in a polymer.

2. The nanoparticulate formulation of claim 1, wherein the pharmaceutical agent is an mTOR inhibitor.

3. The nanoparticulate formulation of claim 1, wherein the pharmaceutical agent is rapamycin.

4. The nanoparticulate formulation of claim 1, further comprising an antioxidant pharmaceutical agent.

5. The nanoparticulate formulation of claim 1, further comprising the pharmaceutical agent 4-hydroxy-[2,2,6,6-tetramethylpiperidin-1-yl]oxidanyl.

6. The nanoparticulate formulation of claim 1, wherein the nanoparticles comprise rapamycin and 4-hydroxy-[2,2,6,6-tetramethylpiperidin-1-yl]oxidanyl encapsulated in a polymer.

7. The nanoparticulate formulation of claim 1, wherein the polymer is a triblock copolymer.

8. The nanoparticulate formulation of claim 7, wherein the triblock copolymer is a poloxamer.

9. The nanoparticulate formulation of claim 8, wherein the poloxamer is poloxamer 188.

10. The nanoparticulate formulation of claim 1, wherein the one or more pharmaceutical agents are conjugated to folate.

11. The nanoparticulate formulation of claim 1, wherein one of the one or more pharmaceutical agents are conjugated to folate.

12. The nanoparticulate formulation of claim 1, wherein more than one of the one or more pharmaceutical agents are conjugated to folate.

13. A method of treating a chronic kidney disease comprising administering to a subject in need thereof a nanoparticulate formulation of claim 1.

14. The method of claim 13, wherein the chronic kidney disease is polycystic kidney disease.

15. The method of claim 13, wherein the nanoparticulate formulation is administered less frequently than daily.

16. The method of claim 15, wherein the nanoparticulate formulation is administered weekly.

17. A method of targeting one or more pharmaceutical agents to an organ of a subject in need thereof, comprising administering to a subject in need thereof a nanoparticulate formulation, the nanoparticles comprising one or more pharmaceutical agents encapsulated in a polymer.

18. The method of claim 17, wherein the pharmaceutical agent is selected from the group consisting of an mTOR inhibitor, an antioxidant, a phosphatidylinositol-3 kinase-related kinases (PIKKs) inhibitor, an ATP-competitive mTOR kinase inhibitor, an mTOR/P13K dual inhibitor, and an mTORC1/mTORC2 dual inhibitor.

19. The method of claim 17, wherein the pharmaceutical agent is selected from the group consisting of rapamycin, a rapalog, temsirolimus, everolimus, ridaforolimus, umirolimus, zotarolimus, torin-1, torin-2, vistusertib, dactolisib, voxtalisib, BGT226, SF1126, AZD8055, AZD2014, OSI-027, INK-128, MLN0128, VX970, NVP-BEZ235, AZ20, AZ31, PKI-587, 4-hydroxy-[2,2,6,6-tetramethylpiperidin-1-yl]oxidanyl (4-hydroxy-TEMPO or TEMPO), metformim, salsalate, oxypurinol, cincalcet, GLPG2737, benzbromarone, niclosamide, senicapoc, pioglitazone, 2-deoxy-D-glucose, a SGLT2 inhibitor, pemafibrate, bardoxolone, sulforaphane, probucol, emalipretide, valproic acid, nicotinamide, and tolvaptan.

20. The method of claim 17, wherein the organ is selected from the group consisting of kidney, lung, ovary, pancreas, and vascular system.
